(19) United States
(12) Patent Application Publication
(10) Pub. No.: US 2003/0233053 A1
(43) Pub. Date: Dec. 18, 2003

(54) Pain assessment

(76) Inventors: Clifford J. Woolf, Newton, MA (US); Joachim Scholz, Charlestown, MA (US); Isabelle Veronique Decosterd, Pully (CH); Salahadin Abdi, Lexington, MA (US)

Correspondence Address: FISH & RICHARDSON PC, 225 FRANKLIN ST, BOSTON, MA, 02110, US

(21) Appl. No.: 10/171,882

(22) Filed: Jun. 14, 2002

(51) Int. Cl. A61B 19/00

(52) U.S. Cl. 600/553

(57) ABSTRACT

Methods and questionnaires are provided for assessing pain.

### TECHNICAL FIELD

This description relates to pain assessment.

### BACKGROUND

Conventionally, pain syndromes are classified according to the affected anatomical structure, e.g. trigeminal neuralgia, tissue category, e.g. fibromyalgia, or the underlying disease, e.g. cancer pain. They are further divided into acute or chronic conditions related to the duration. Another common division has been to differentiate pain produced by damage to or dysfunction in the nervous system, neuropathic pain, from pain arising from soft tissue, viscera, bone or joints, which have been termed nociceptive or inflammatory pain, depending on the presence or absence of tissue damage and inflammation. There have been attempts to assign specific pain qualities to these different clinical diagnoses. However, the list of qualities attributed to conditions of neuropathic pain alone includes descriptions as diverse as burning, shooting, crawling pain, unusual tingling, electrical sensation or sharp, pulling, aching, tender pain and such descriptors provide little insight into the mechanisms underlying the induction or maintenance of the pain.

There are several lines of evidence indicating that it is not possible to regard pain in general, or even a particular diagnostic disorder like postherpetic neuralgia, as a uniform entity. The relation between etiology, underlying mechanisms and the specific symptoms and signs related to painful disorders is complex. Pain in an individual patient may be produced by more than one mechanism. No pain mechanism is the inevitable result of a particular etiology. Finally, different mechanisms may operate in patients with the same disease and these may even change during the course of the disease.

Current strategies for assessing pain severity or the efficacy of a given treatment against pain generally rely on global outcome measures. Patients are asked to rate the overall intensity of their pain on categorical scales ranging from no pain to severe pain. Also widely used are numerical rating scales from 0 to 10, where 10 stands for the worst possible pain, or visual analogue scales, where pain intensity has to be indicated on a horizontal line or similar depiction scaled from 0 to 100. Other, secondary, measures of pain intensity are based on the consumption of analgesic drugs or pain-related deficits in function or the quality of life.

### SUMMARY

In one aspect, the invention features a method including accumulating information from a patient regarding a pain or other sensation experienced by the patient, the information being selected to be indicative of at least one mechanism associated with the pain or sensation, and analyzing the information to determine one or more pain mechanisms indicated by the information.

Some implementations may include one or more of the following features. The method further includes deriving a pain profile based on the information. The method further includes analyzing the pain profile to determine the presence of at least one pain feature. The pain mechanism is determined based on the state of the pain feature. The information includes the location of the pain or sensation as indicated on an anatomical diagram. The information includes the depth of the pain relative to the skin surface of the patient. The information pertains to a single pain. The information pertains to two or more separate pains and/or sensations the information being selected to be indicative of one or more than one mechanism associated with the pains and/or sensations. The information includes how long ago the pain or sensation started. The information includes the time course of the pain or sensation, e.g., changes of the pain or sensation over time. The information includes the temporal characteristics of the pain or sensation, e.g., the time profile of instances of the pain or sensation. The information pertains to a sensation other than pain. The information pertains to events that evoke a pain or other sensation, e.g., mechanical or thermal stimuli. The information pertains to the quality of the pain. The information pertains to factors that alleviate a pain or other sensation experienced by the patient. The information pertains to the presence of a sensory deficit. The information is accumulated using a questionnaire and/or by eliciting the patient's history using standardized questions. The information is accumulated by performing a physical examination according to a standardized protocol. The method further includes diagnosing a pain syndrome based on the mechanisms determined. The method further includes treating the patient based on the mechanisms determined. The method further includes analyzing the information along with information accumulated from other patients.

In another aspect, the invention features a method including accumulating information from a patient regarding a pain or other sensation experienced by the patient, the information being selected to be indicative of at least one mechanism associated with the pain or sensation, and graphically or numerically representing a presence, relative amplitude, and absence of individual symptoms and signs to create a pain profile for the patient.

The invention also features a method including accumulating information from a patient regarding a pain or other sensation experienced by the patient, the information being selected to be indicative of at least one mechanism associated with the pain or sensation, and identifying from the information composites of symptoms and signs that represent pain features that are indicative of a pain mechanism.

In a further aspect, the invention features a method including accumulating information from a patient regarding a pain or other sensation experienced by the patient, the information being selected to be indicative of at least one mechanism associated with the pain or sensation, and performing a therapeutic evaluation of a patient based on the information.

In some implementations, the method also includes, at a later time, accumulating additional information from a patient regarding a pain or other sensation experienced by the patient, the information being selected to be indicative of at least one mechanism associated with the pain or sensation, and, performing a diagnostic or therapeutic evaluation of the patient based on the additional information.

In yet another aspect, the invention features a method including accumulating information from patients regarding pains or other sensations experienced by the patients, the information being selected to be indicative of at least one mechanism associated with the pains or sensations, and performing an epidemiological study with respect to the mechanisms associated with the pains or sensations based on the accumulated information.

The invention also includes a method including accumulating information from patients regarding pains or other sensations experienced by the patients, the information being selected to be indicative of at least one mechanism associated with the pains or sensations, selecting or modifying a drug under development based on the accumulated information, testing the drug on the patients, accumulating additional information from the patients regarding pains or other sensations experienced by the patients, the information being selected to be indicative of at least one mechanism associated with the pains or sensations, and iterating the testing and accumulating until a marketable drug is reached.

Among other advantages of the invention are one or more of the following. Pathophysiological targets may be identified for rational, non-empirical treatment in individual patients. Pain conditions of a variety of etiologies may be addressed: traumatic, inflammatory and those associated with a structural lesion or malfunction of the nervous system.

Other features and advantages of the invention will be apparent from the description and drawings, and from the claims.

### DESCRIPTION OF DRAWINGS

FIG. 1 is a flow chart comparing a conventional approach to pain management with a mechanism-based approach.

FIG. 1A is a flow chart illustrating steps of the mechanism-based approach of FIG. 1.

FIG. 2 is a block diagram illustrating how single symptoms and signs present in conditions associated with spontaneous neuropathic pain are common to multiple clinical diseases and pain syndromes.

FIG. 3 is a table illustrating the relationship between pain mechanisms and symptoms and signs.

FIG. 4 demonstrates, by giving two examples, that pain features are composites of pain symptoms and signs.

FIG. 5 is an illustration of pain profiles produced by examples of different pain mechanisms, showing the pain features typical of these mechanisms.

FIG. 6 shows an example of a mechanism-based assessment of pain.

FIG. 6A is a block diagram of a computer system that is suitable for use in mechanism-based pain assessment.

FIG. 7 is a table providing an overview of examples of treatment strategies based on pain mechanisms.

FIG. 8 is a flow diagram illustrating an example of steps used in assessing a patient's pain.

FIGS. 9 and 10 are diagrammatic representations of methods according to two alternative embodiments of the invention.

FIGS. 11A-11J are pages of an example of a Patient History Questionnaire.

FIGS. 12A-12H are pages of an example of a Physical Examination Questionnaire.

### DETAILED DESCRIPTION

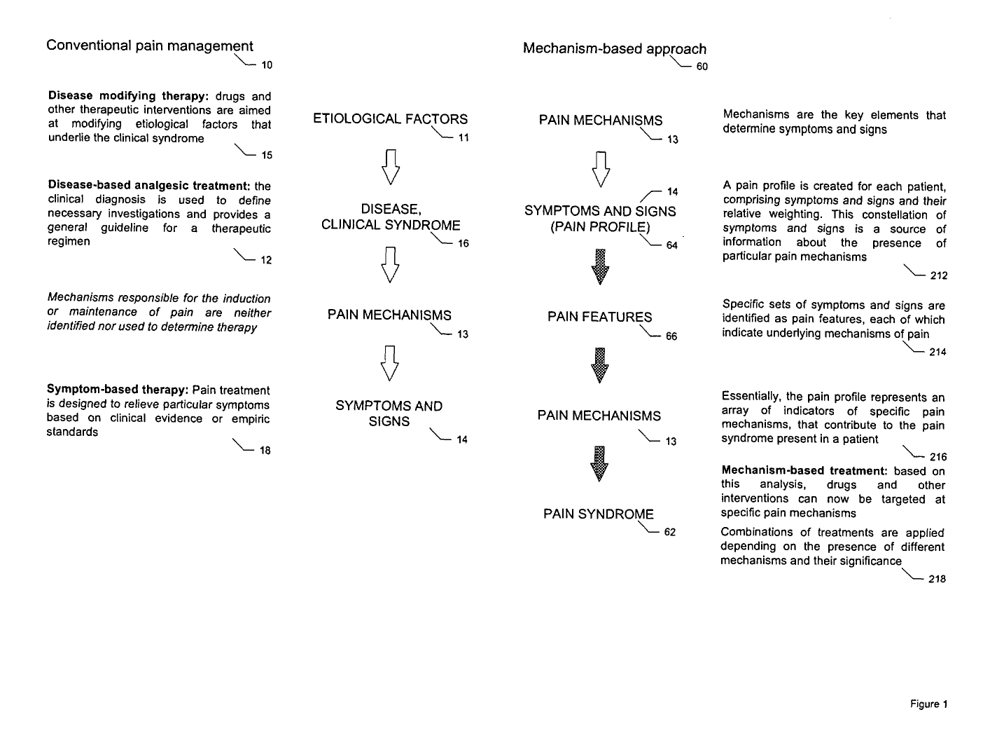

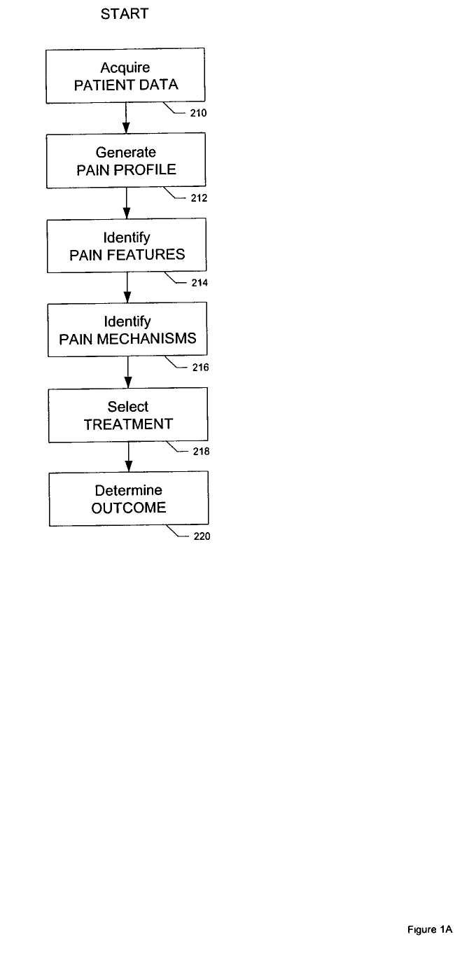

As shown in the two left-hand columns of FIG. 1, conventional management of pain (10) generally relies on clinical diagnoses of diseases and clinical syndromes (16), for example rheumatoid arthritis and herpes zoster, and on knowledge of the underlying etiological or causative factors (11), i.e., in these examples an autoimmune reaction and a viral infection, respectively. The treatment would be aimed at (a) modifying the underlying etiological factors (11) (i.e., disease-modifying therapy (15)), (b) prescribing an analgesic based on empiric standards (i.e., symptom-relief therapy (18)), or (c) a combination of these therapies (12). In this conventional approach, the pain mechanism (13), which actually drives the pain in a patient, would not be identified nor would it be the target of the therapeutic intervention.

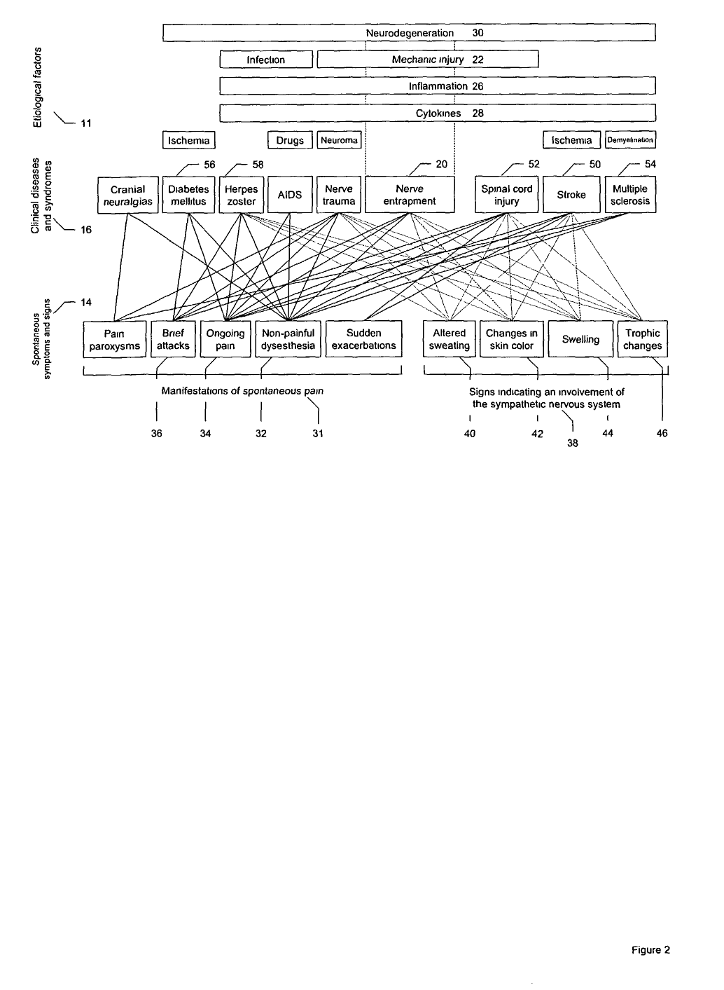

However, as illustrated in FIG. 2 for conditions that are typically associated with spontaneous neuropathic pain, individual symptoms and signs (14) are common to many different clinical diseases or syndromes (16). Thus, neither a disease diagnosis nor individual symptoms alone can be used to identify the mechanisms responsible for the production of the pain.

As an example, again referring to FIG. 2, the clinical syndrome of nerve entrapment (20) is associated not only with the etiological factor of mechanical compressive injury (22) but also with other etiological factors (11) such as inflammatory changes (26) or the release of cytokines (28) at the site of the injury, or neurodegeneration (30) if untreated, as well as with a possible involvement of the sympathetic nervous system. (Examples of etiological factors (11) are depicted on top of FIG. 2; the lateral extension of the boxes indicates the relevance of each etiological/causative factor for the clinical diseases or syndromes below, as indicated by dotted lines in FIG. 2 for the nerve entrapment example).

The relationship between clinical diseases and syndromes (16), and spontaneous symptoms and signs (14), is indicated in FIG. 2 by the lines connecting individual diseases/syndromes with the associated symptoms and signs below. Again using nerve entrapment as an example, spontaneous symptoms (14) associated with nerve entrapment include manifestations of spontaneous pain (31), non-painful but unpleasant, disturbing sensations (so-called parasthesia or dysesthesia (32)), ongoing pain (34) or pain that is intermittently present in brief attacks (36). Especially in those cases when the median nerve is entrapped at the wrist, a condition called carpal tunnel syndrome, signs indicating an involvement of the sympathetic nervous system (38) can be found in the physical examination. Altered sweating (40) and a bluish or otherwise changed skin color (42) are indicators of a dysfunction in the sympathetic nervous system, while swelling (44) and trophic changes of the skin or the nails (46) represent consequences of disturbances in the blood supply.

As illustrated in FIG. 2, symptoms and signs attributable to a dysfunction in the sympathetic nervous system (38), which can be present in patients with nerve entrapment, as discussed above, can also be present in patients with other diseases, e.g., herpes zoster (58), when the pain that accompanies the acute virus infection turns into a chronic condition named postherpetic neuralgia. Furthermore, symptoms and signs indicative of an involvement of the sympathetic nervous systems may be observed in diseases and injuries that affect structures of the central nervous system, e.g., the brain with stroke (50), the spinal cord after injury (52), and the brain and/or the spinal cord with multiple sclerosis (54). Similarly, the same symptoms and signs involving manifestations of spontaneous pain (31) that are present in patients with nerve entrapment can also be present in patients with other diseases, such as diabetes mellitus (56) and herpes zoster (58).

Pain mechanisms are those factors operating within the sensory nervous system that are responsible for producing pain symptoms including spontaneous and evoked pain. Different diseases or clinical syndromes may initiate different mechanisms, or the same mechanism, and the same disease or clinical syndrome may generate pain by different mechanisms, either between patients, or even in the same patient over the course of the disease. Thus, patients may have pain mechanisms in common even though their clinical diagnoses are different. For example, a reduced threshold of nociceptive sensory neurons may result from joint inflammation in one patient with, e.g., rheumatoid arthritis, or it may be caused by a reactivation of the varicella-zoster virus in another patient.

A disease or clinical syndrome (16) (e.g. painful polyneuropathy in diabetes mellitus, or postherpetic neuralgia developing after herpes zoster) is not equivalent, therefore, to a pain syndrome (62, FIG. 1) (e.g., postoperative pain, inflammatory pain, fibromyalgia, central pain, deafferentation pain), which is a particular pattern or constellation of pain mechanisms (13). It is useful for a rational approach to the diagnosis and treatment of pain that pain mechanisms be identified and form the basis for a mechanism-based approach (60) to determining treatment strategies, as shown in the two right-hand columns of FIG. 1.

In some implementations, a patient's pain mechanisms are identified by first identifying a cluster of multiple, pain-related symptoms and signs, which we call a “pain profile” (64), for the patient. Pain-related symptoms are subjective changes noticed or reported by the patient, e.g. pain provoked by a specific movement like walking or the temporal characteristics of pain attacks. Pain-related signs are objective findings that can be obtained by a clinician examining the patient, e.g. signs of inflammation like reddening or swelling. The pain profile represents a distinct pattern or “fingerprint” of the patient's pain, displaying all the pain-related symptoms reported by the patient during an interview and the signs elicited by a physical examination.

Composites of symptoms and signs in the pain profile define “pain features” (66) that each reveal a specific pain mechanism (13) responsible for the development or the maintenance of pain.

For example, a patient suffering from pain caused by an injury of sensory neurons will complain of numbness in the affected skin area, as sensory neurons are the structures in the peripheral nervous system that are responsible for providing information about stimuli applied to the skin, such as touch or pinprick. If sensory neurons are lost, the sensation of touch is impaired.

In the physical examination, sensory loss can be assessed by applying standardized stimuli and recording the patient's response to these stimuli. The composite of the pain characteristics reported by this patient, i.e., the description of numbness, and the sensory loss found upon examination, define a pain feature indicative of pain due to the degeneration of sensory neurons. On the other hand, a patient suffering from pain caused by a reduced threshold of nociceptive sensory neurons, which specifically detect painful stimuli such as pinprick, will complain of an increased sensitivity of the skin, and a physical examination will produce findings that demonstrate an enhanced responsiveness to a pinprick or similar, painful stimuli in the affected area. In this case, the composite of the pain characteristics of this patient defines a pain feature indicative of pain due to a reduced nociceptor threshold.

Thus, as shown schematically in FIG. 1A, the sequence of steps in providing a mechanism-based pain assessment includes: acquiring patient data (210), generating a pain profile for the patient (212), identifying pain feature(s) (214), and, from the pain feature(s), identifying the corresponding pain mechanism(s) responsible for the patient's pain (216). Based on this analysis, a mechanism-based treatment can be selected (218), and the outcome can be assessed using similar methodology (220).

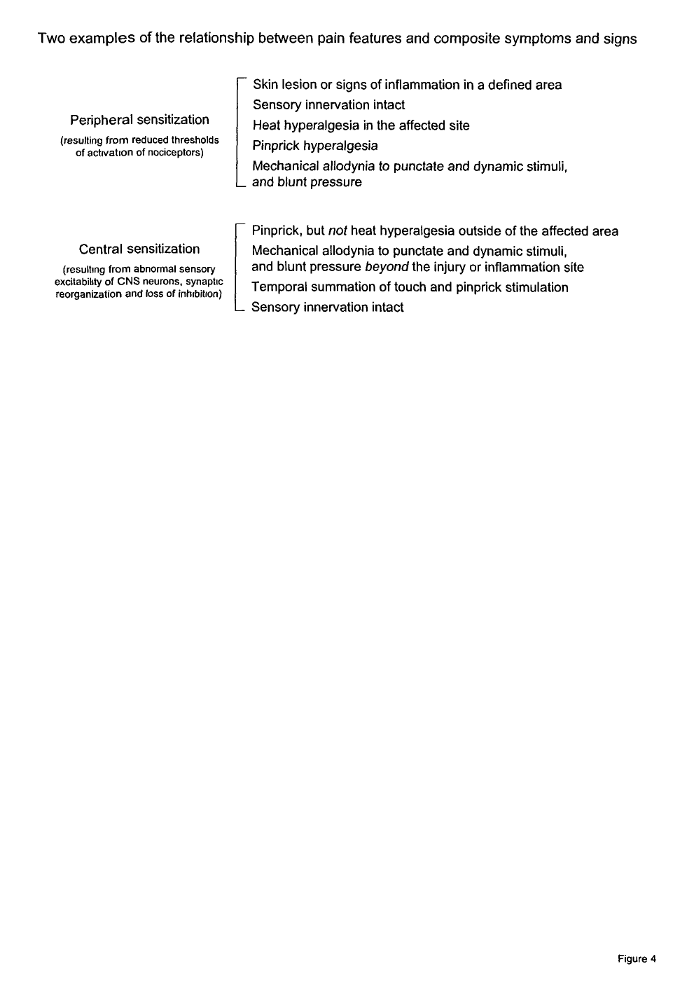

FIG. 4 illustrates examples of the composite symptoms and signs that may make up two different, exemplary pain features. Peripheral sensitization, which is characterized by a reduced activation threshold of nociceptors, is indicated by the following symptoms and signs: skin lesions or local signs of inflammation, an intact sensory innervation, and as decisive indicators, an increased painful response (hyperalgesia) to heat stimuli and pinprick as well as a painful sensation felt upon stimulation with normally non-painful stimuli like punctate, blunt or dynamic mechanical stimuli. These symptoms and signs are confined to the site of the skin lesion or inflammation. The complex pain feature of central sensitization results from abnormal sensory excitability of central nervous system neurons, synaptic reorganization and loss of inhibition. As in peripheral sensitization, it is associated with pinprick but not with heat hyperalgesia. Usually non-painful mechanical stimuli again elicit pain, but this is a finding that goes beyond the skin injury site. In addition, temporal summation, which means an increasing sensitivity to repeated stimuli over time for touch and pinprick stimulation may be present.

Information elicited from the patient is selected to determine the absence or presence of particular symptoms and signs. In addition, the relative amplitudes of individual symptoms and signs are determined by using a rating system such as a categorical scale ranging from none or absent to severe, or a numerical rating scale from 0 to 10. Suitable techniques for eliciting this information will be discussed in detail below. The rating systems are not used only to measure global pain intensity. They are employed to judge the relative contribution of a specific pain symptom or sign to the patient's pain profile. Based on this information, the pain profile enables the determination of the presence or absence of the pain features, and their relative significance for the patient's pain, as will be discussed in further detail below.

The collection of pain-related data may also include findings from special investigations, e.g., testing beyond the scope of the physical examination, like computed tomography (CT) scans of the spine in patients with low back pain, or the results from a laboratory examination of the cerebrospinal fluid and magnetic resonance imaging (MRI) of the brain and the spinal cord in patients with multiple sclerosis.

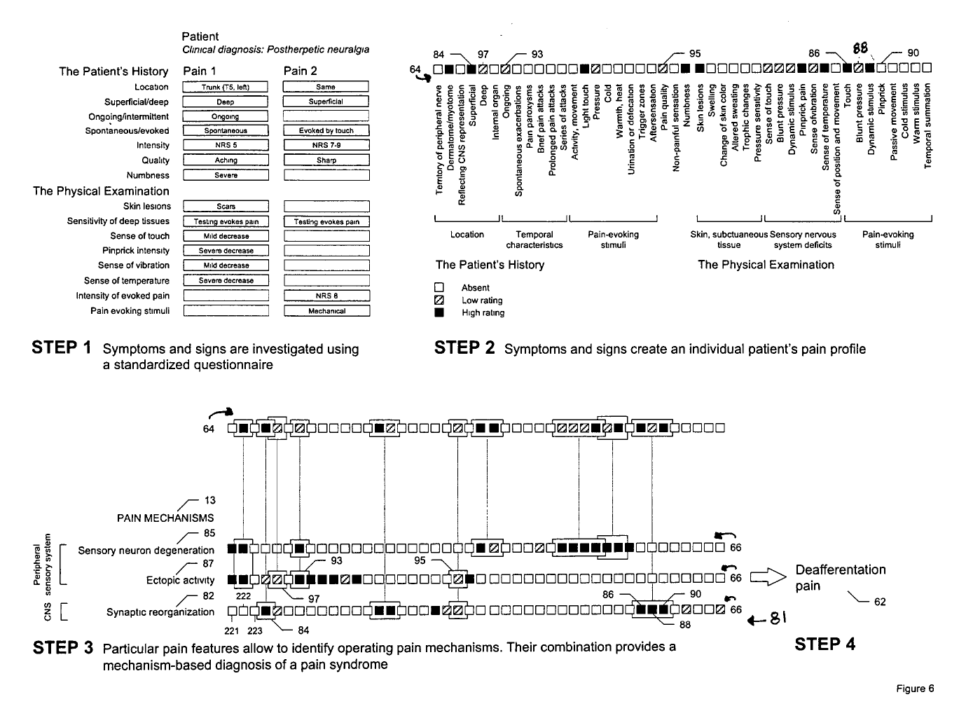

The pain experienced by an individual patient may be caused by more than one pain mechanism (13). For example, a patient's pain profile (64) shown in step 2 of FIG. 6 (discussed in detail below) indicates that the patient's pain is caused by sensory neuron degeneration (85), ectopic activity (87), and synaptic reorganization (82) (step 3, FIG. 6).

Since multiple mechanisms may co-exist, the pain profile may contain one or more than one pain feature. In a stepwise analysis, the distinctive pattern of symptoms and signs is used first, to identify those pain features, and next, to conclude on the single or multiple pain mechanisms present. This is achieved by comparing the pain profile of the individual patient with a database (e.g., databases 70 and 73, FIG. 6A) that is created using the same mechanism-based tool for the assessment of pain from a large population of patients, including those where single mechanisms can be shown or inferred to be present and associated with particular pain features. As described in detail below, the database 73 includes a collection of pain features associated with single pain mechanisms and provides a key for decoding a patient's pain profile to reveal the most likely or predominant mechanism responsible for his pain, or a complex combination of mechanisms that may co-exist in the patient and constitute a pain syndrome. Matches of the individual patient's pain profile with single mechanism-specific pain features allow detection of the presence of the corresponding pain mechanisms. As a final result, a mechanism-based diagnosis of the individual patient's pain syndrome is obtained.

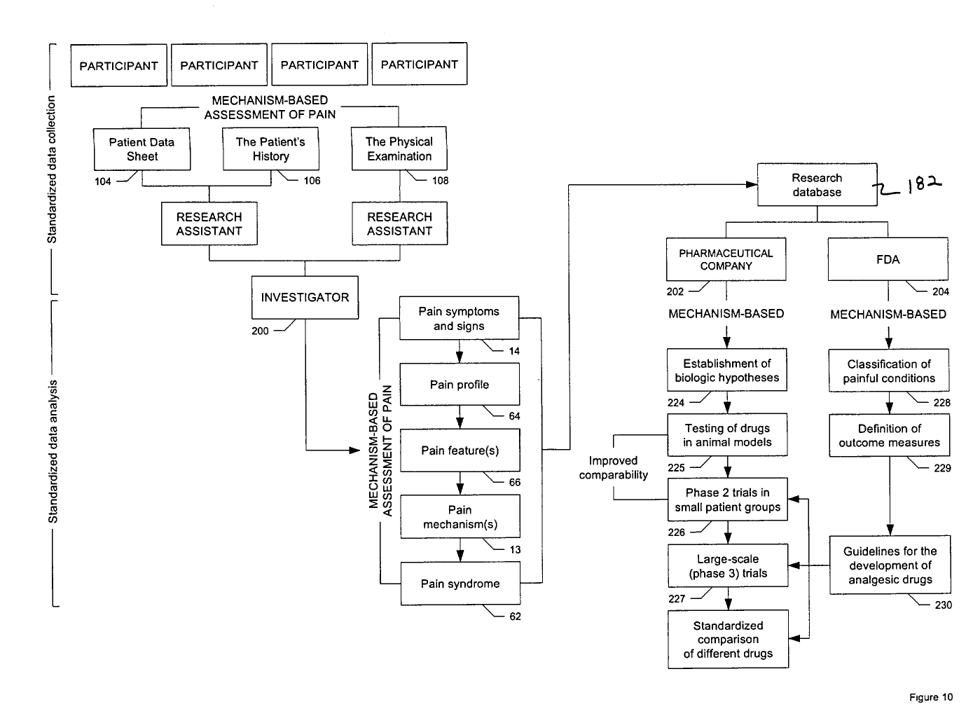

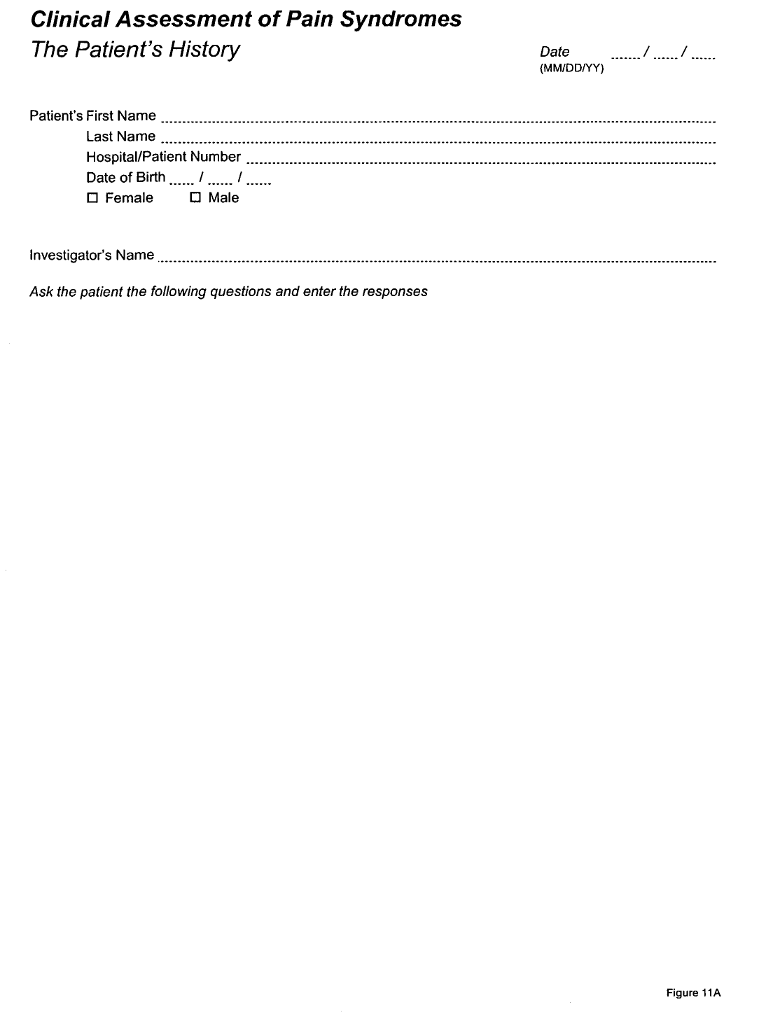

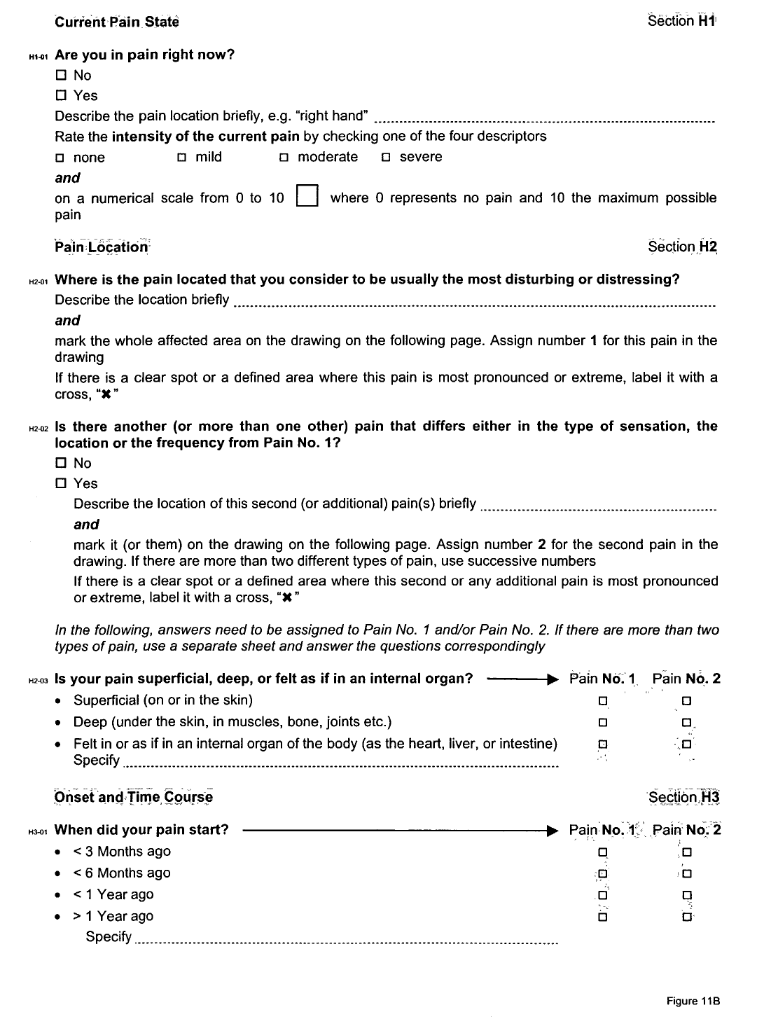

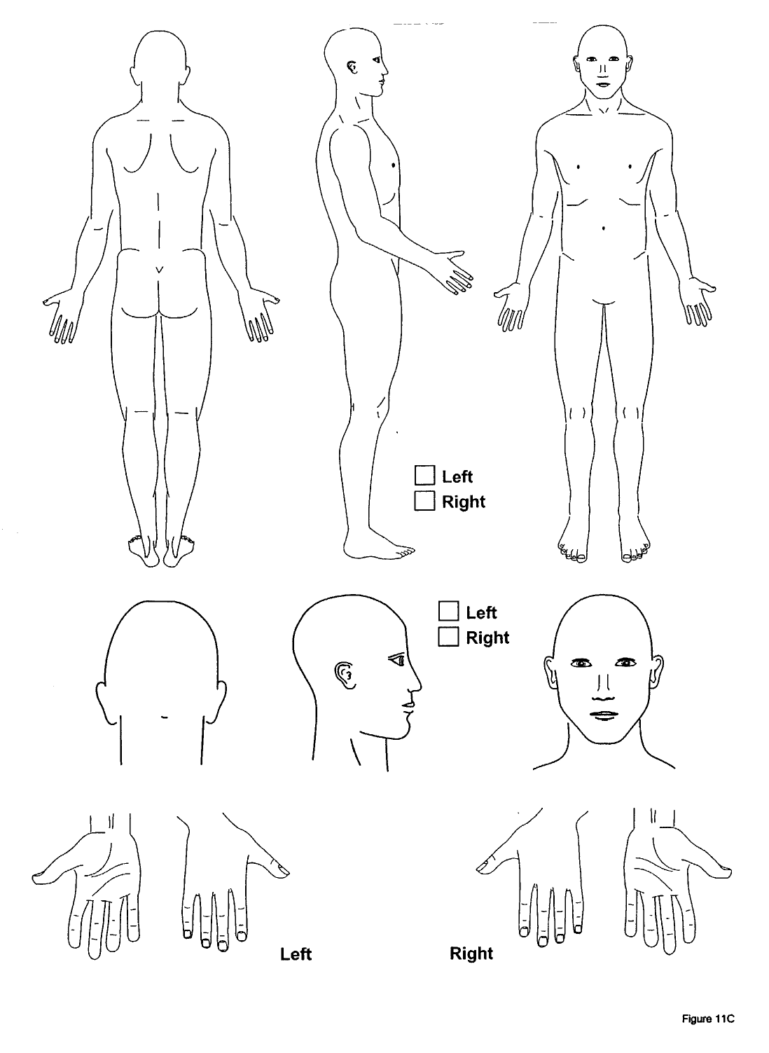

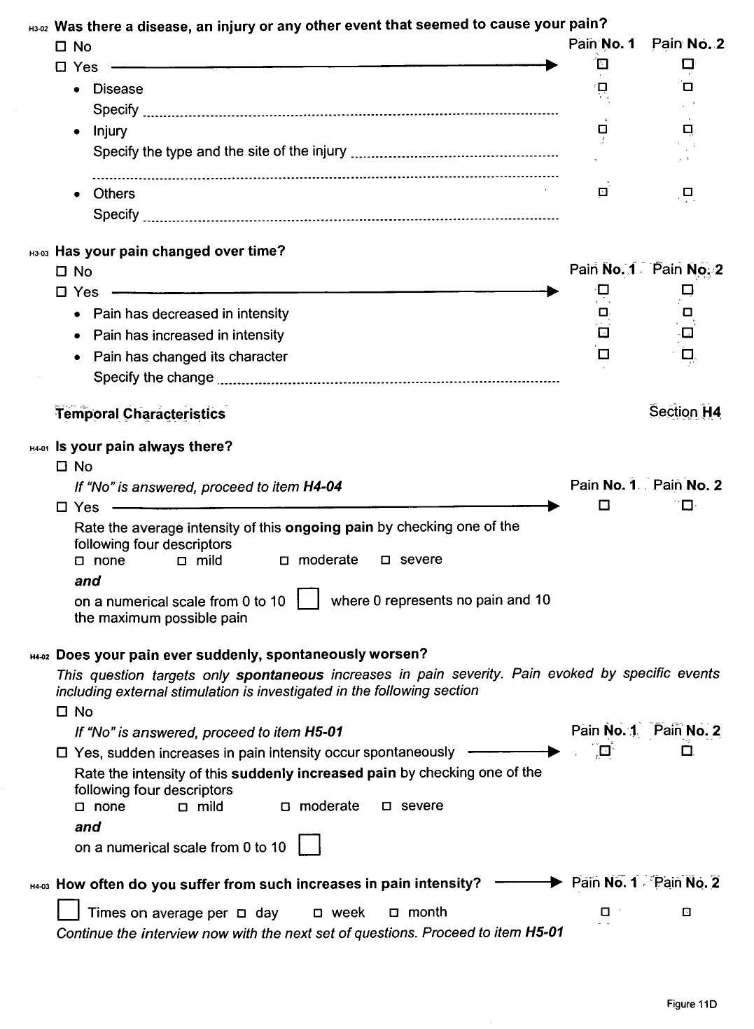

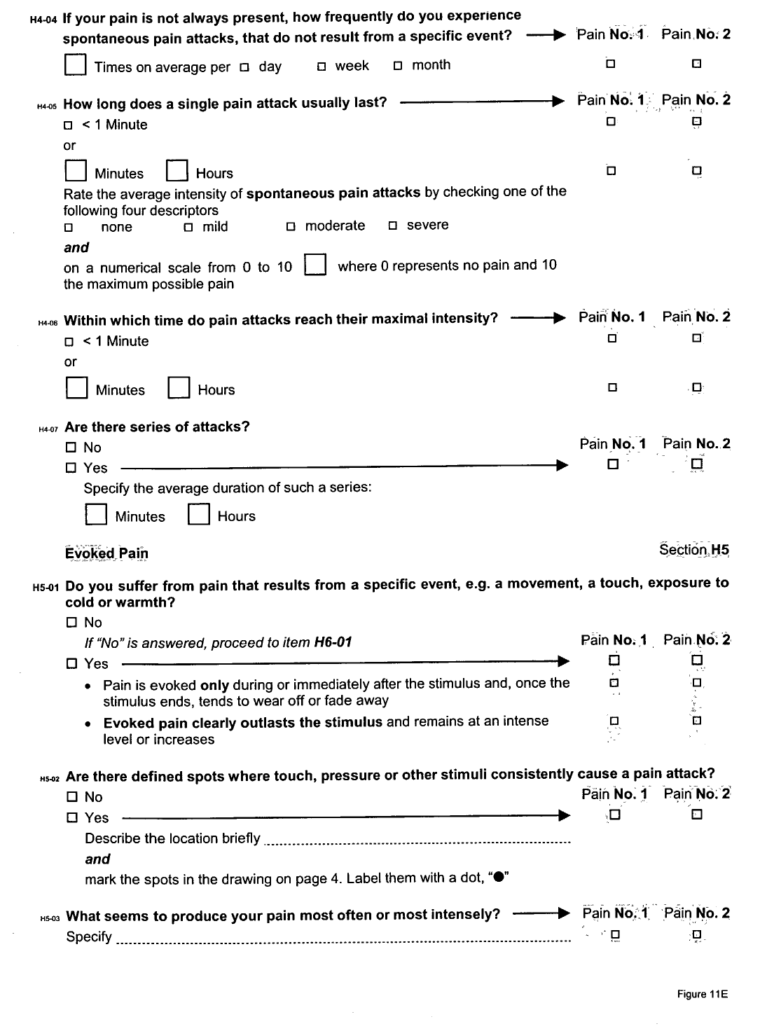

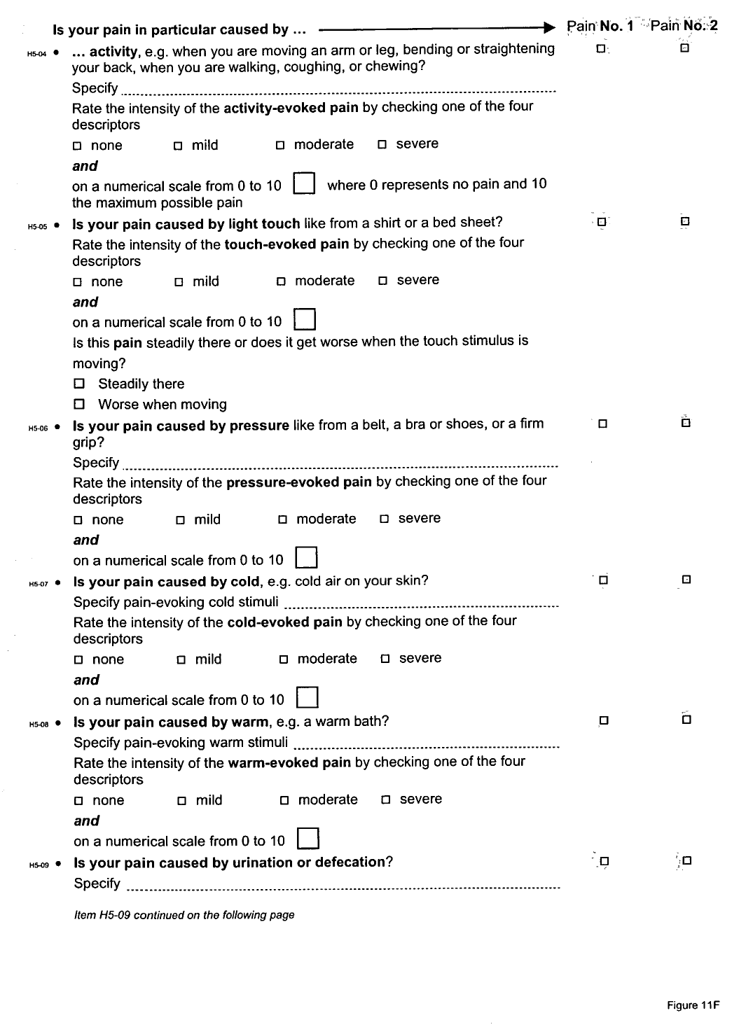

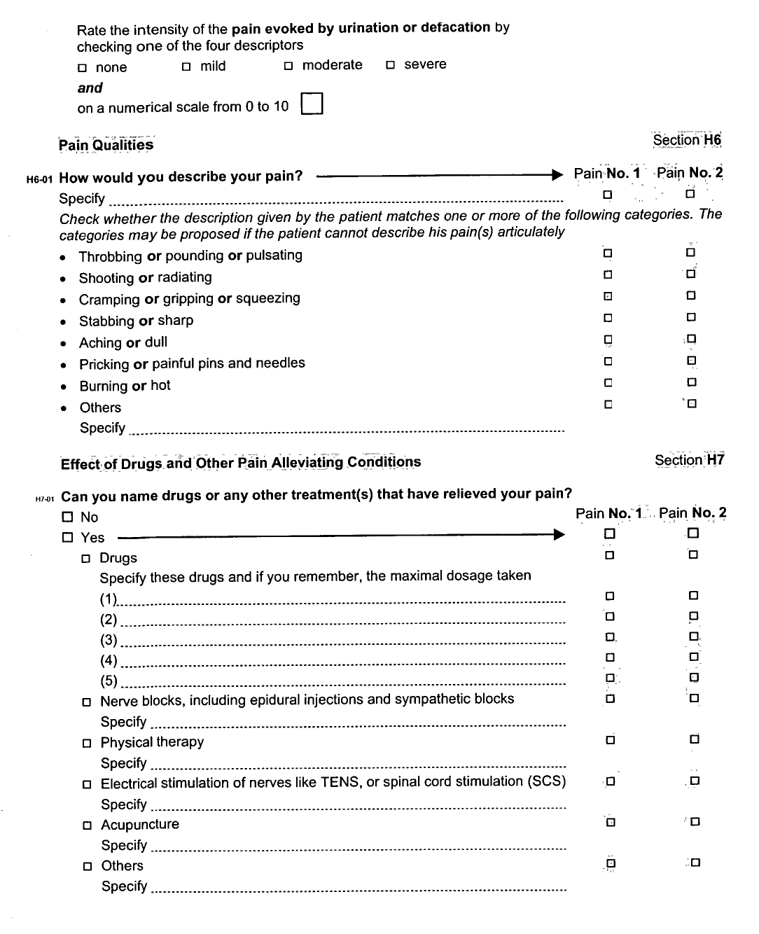

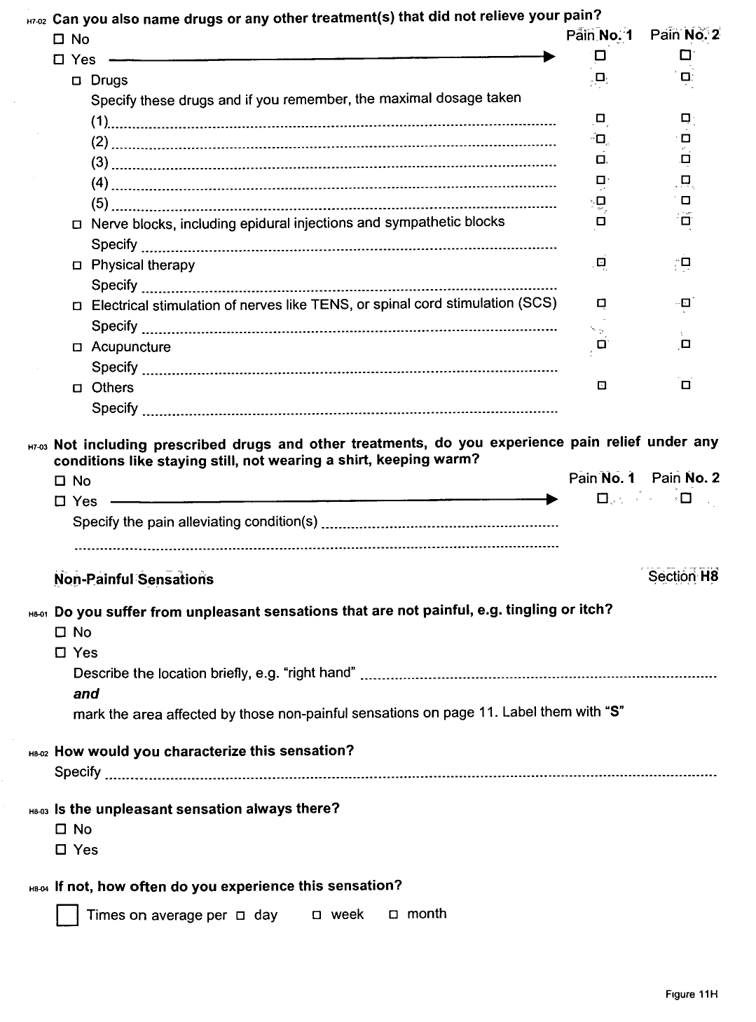

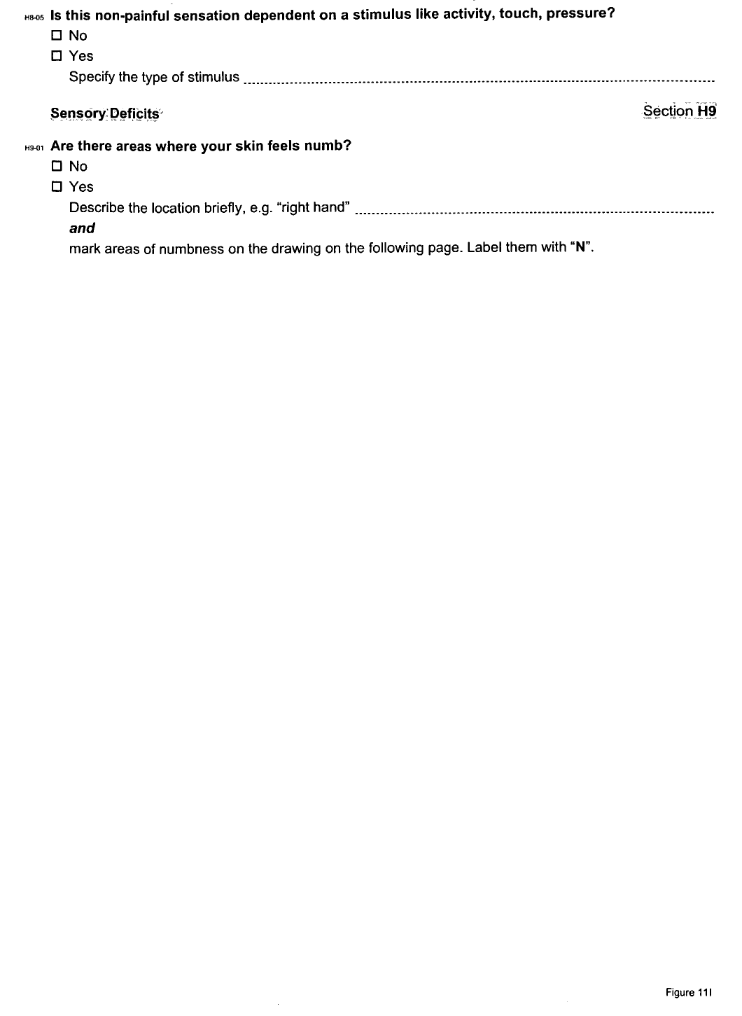

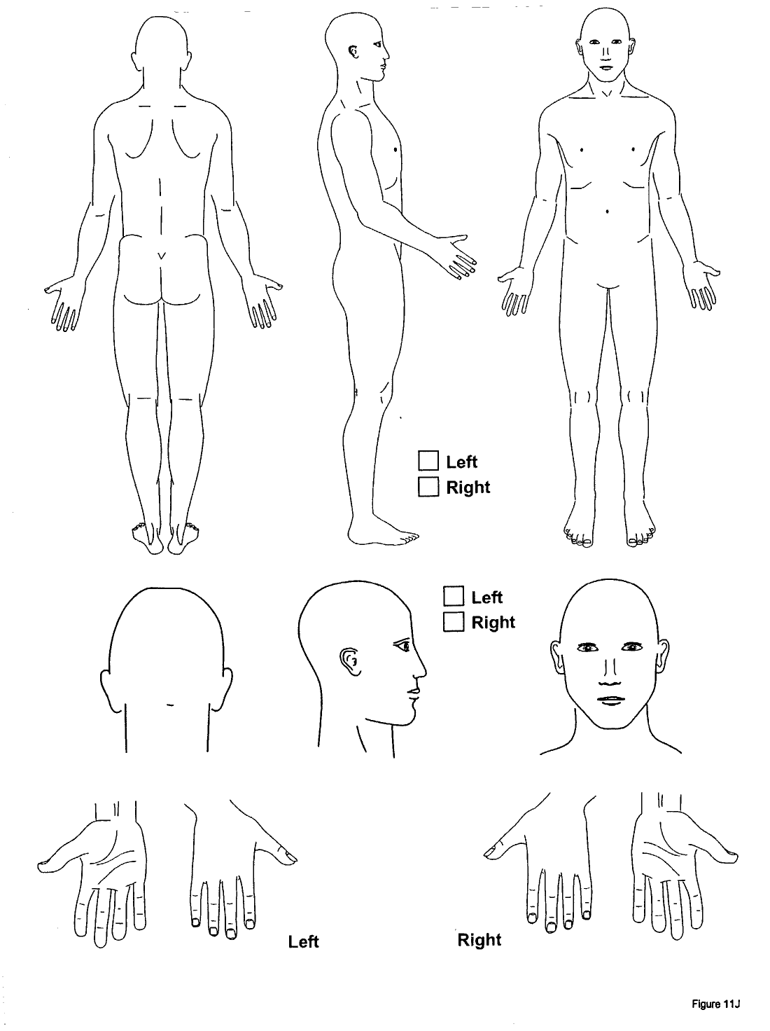

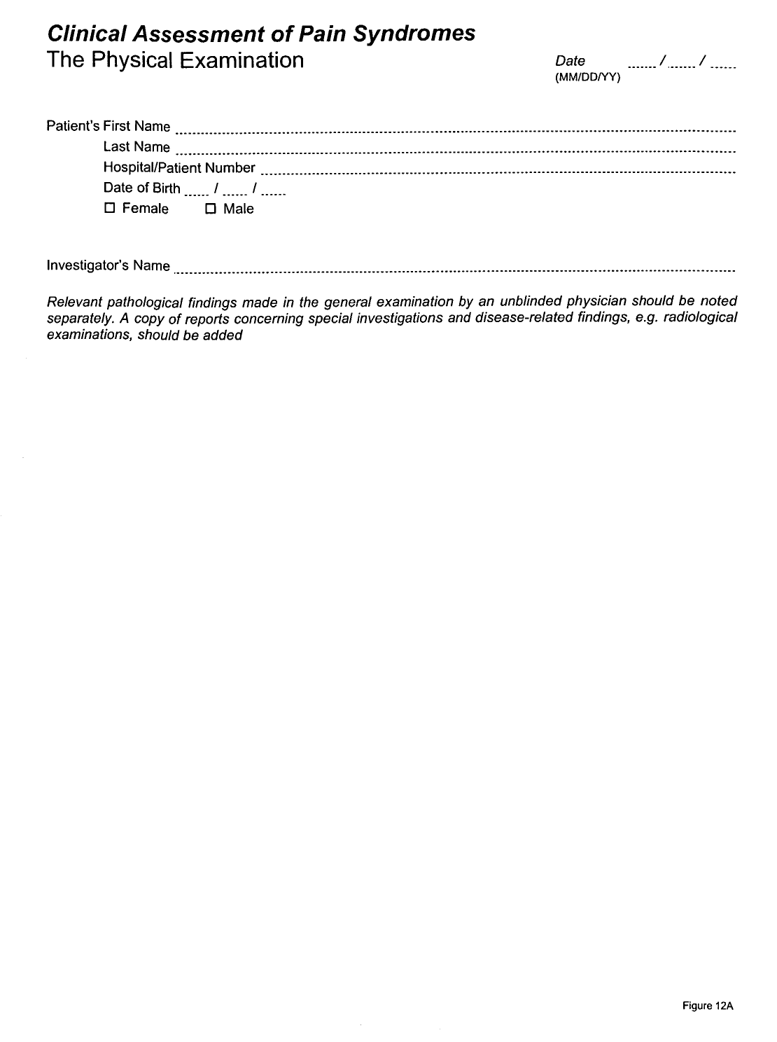

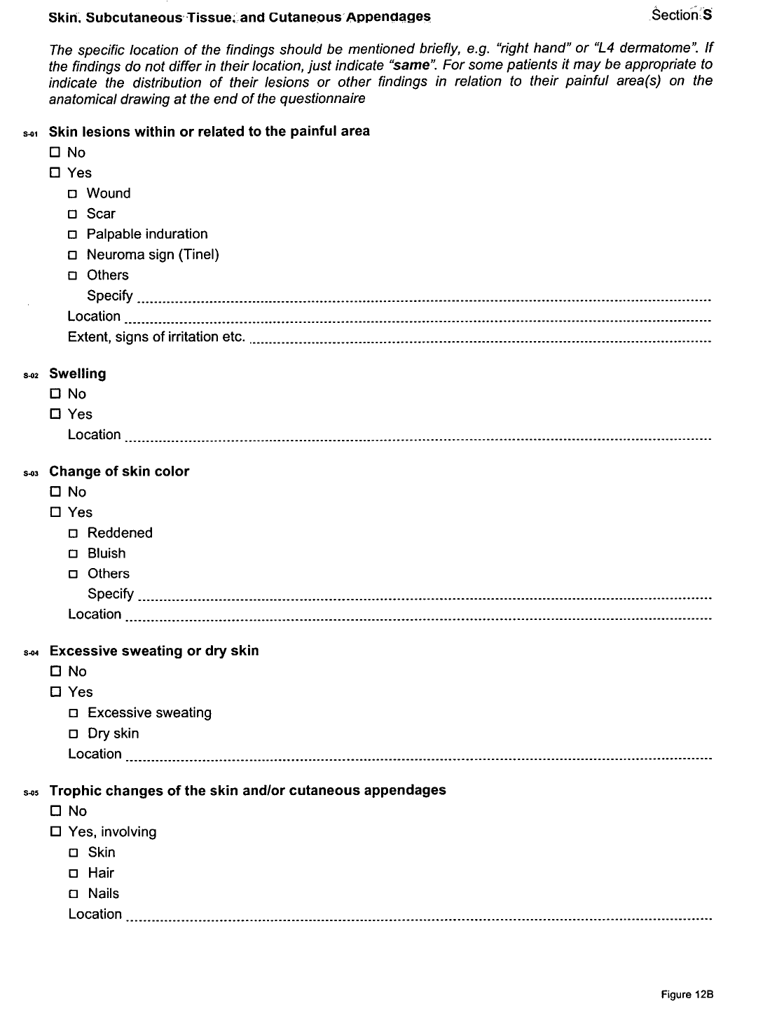

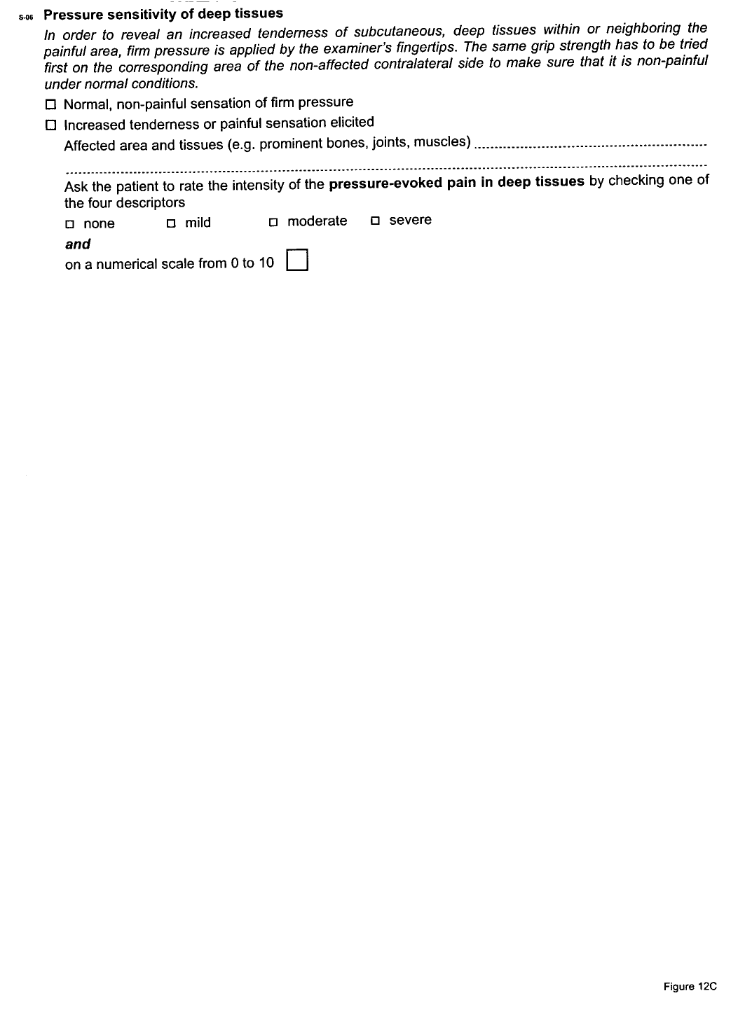

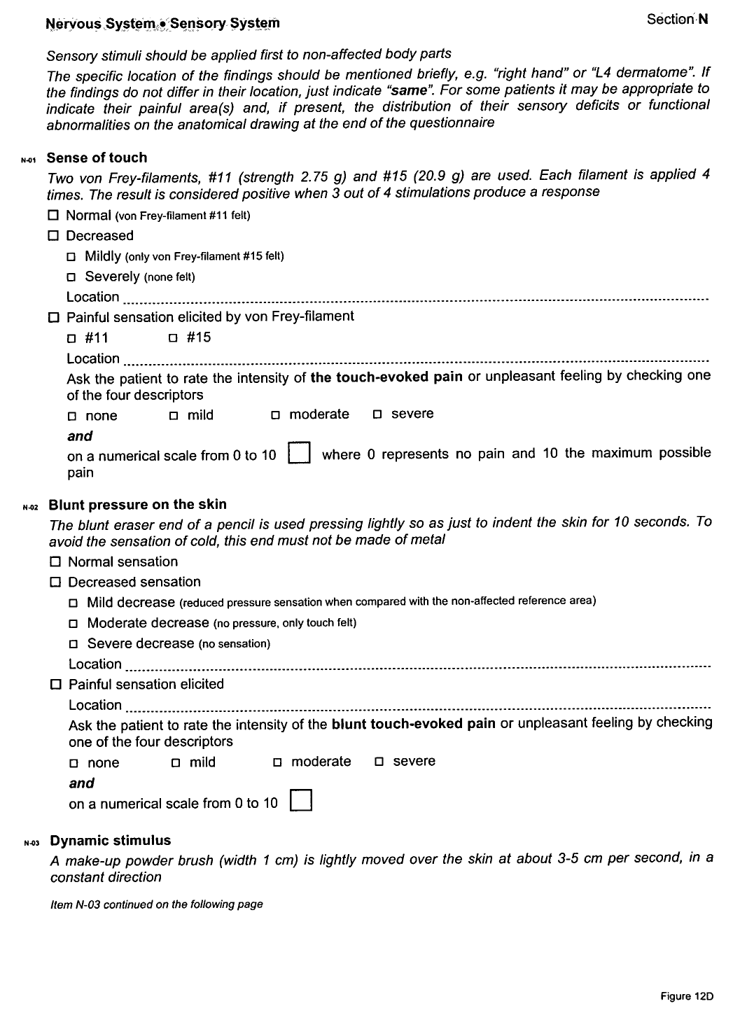

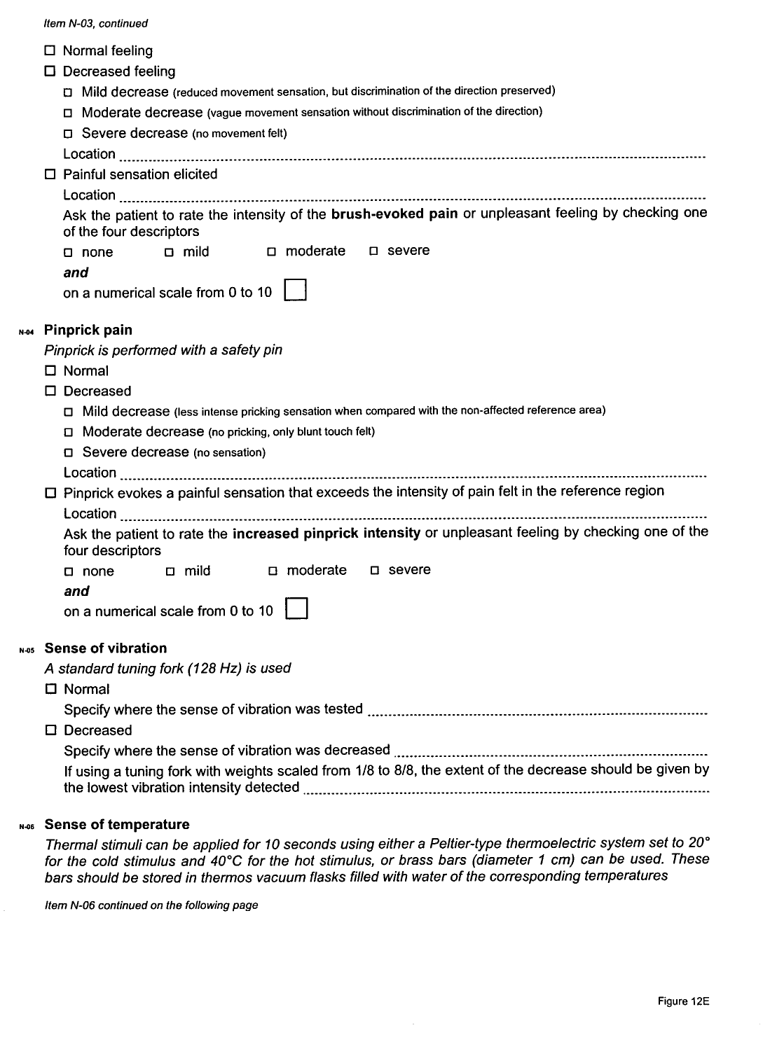

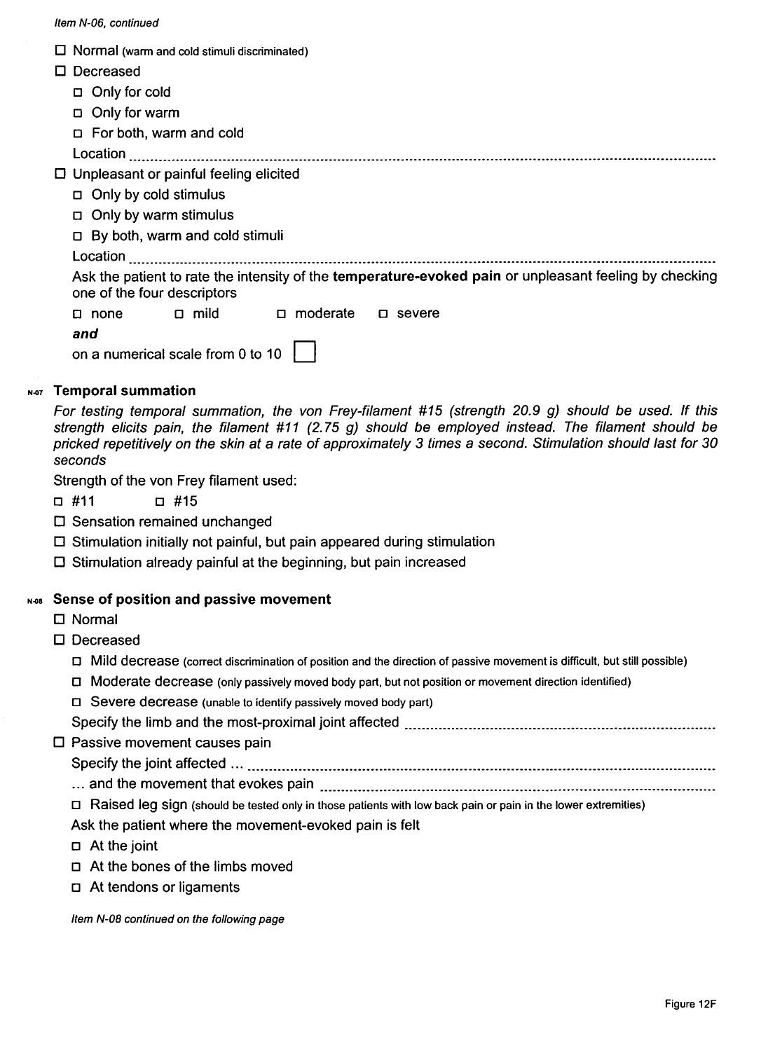

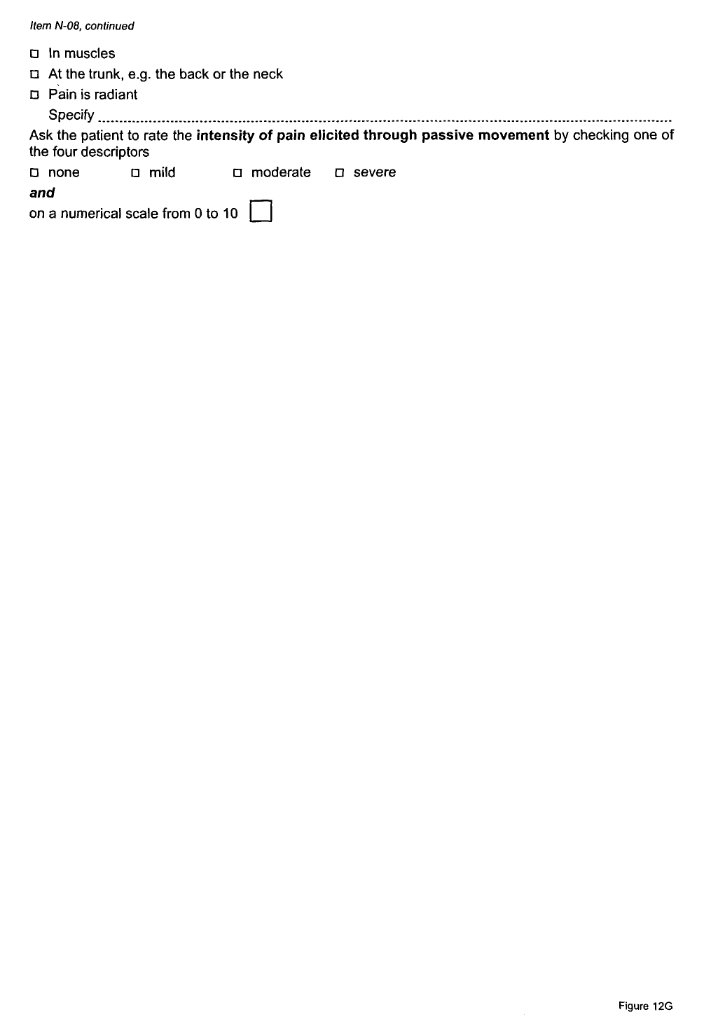

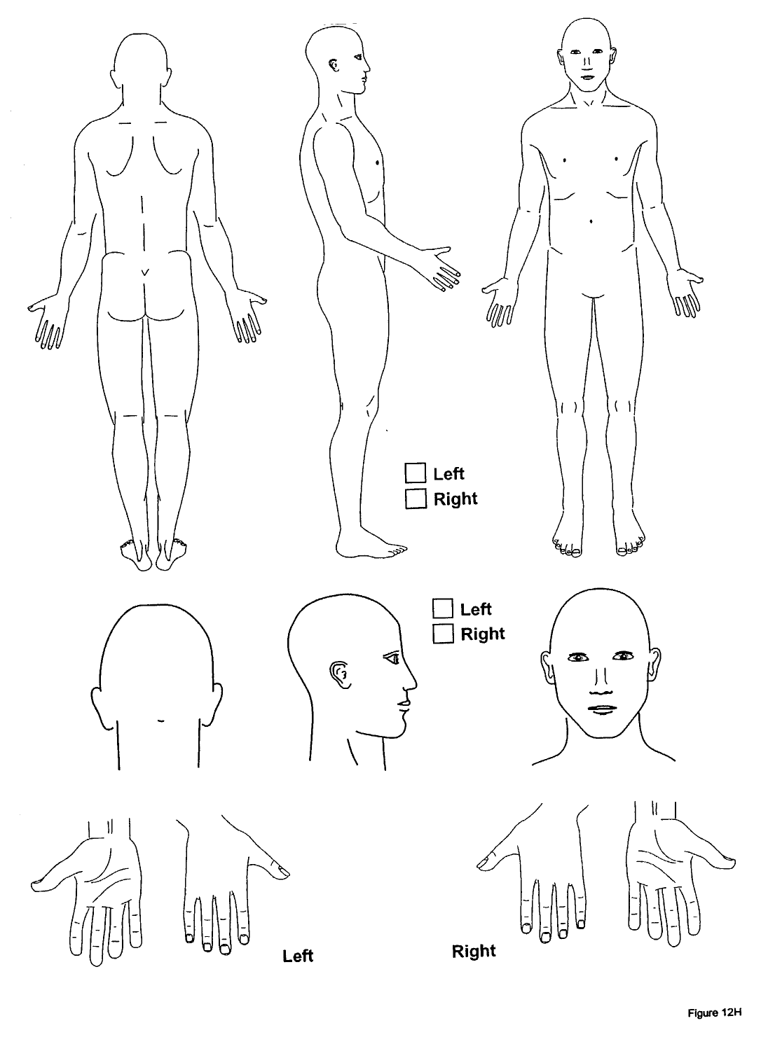

In some implementations, a questionnaire on the patient's history (e.g., as shown in FIGS. 11A-11J and discussed below), a physical examination guided by standardized instructions (e.g., as shown in FIGS. 12A-12H), and, where appropriate, special investigations, are used to elicit information from the patient. The questionnaire and the physical examination are used to investigate the presence of spontaneous and evoked pains, temporal and spatial evolution of the symptoms, localization, distribution and nature of evoked pain, and other sensory abnormalities and associated changes. The questionnaire and the standardized instructions for the physical examination are designed to provide a detailed pain profile, which is comprehensive, precise, and reflects the relative contribution of each symptom and sign according to a rating scale.

This mechanism-based assessment of pain will have consequences for the management of pain. Predominant mechanisms of pain may be identified in a given patient and drugs that are known to target the symptoms generated by these mechanisms can be selected for treatment of the patient. Use of the questionnaire and the standardized examination described below, by enabling the pain profile of the patient to be established, will allow clinicians to standardize investigation and documentation of both initial findings and findings regarding improvement or worsening of a pain syndrome during follow-up visits. Changes in the mechanisms responsible for the pain over the course of a disease can be followed by the appearance or disappearance of different pain features in the pain profile or relative changes in the rating of correlated symptoms and signs.

In addition, the development and the evaluation of new analgesic drugs will be facilitated. Mechanism-based pain assessment may improve the design of clinical trials, by allowing the investigation of drug effects on specific mechanisms of pain, rather than attempting to measure overall reduction of pain. Recent evidence from fundamental pharmacological research has revealed previously unknown mechanisms of drug action. These include, for example, the effect of cyclooxygenase (COX)-2 inhibitors on the central nervous system, and the binding of gabapentin to the α2δ-subunit of voltage-gated calcium channels (VGCC), which is up-regulated in animal models of neuropathic pain. Mechanism-based pain assessment will also provide tools to select study populations of patients with pain based on underlying mechanisms. Eventually, mechanism-based assessment of pain may radically change the design of pharmacological trials to study specific effects of drugs on pain mechanisms. Likewise, it will have an important impact on the design of investigations involving the significance of known modem of drug action as well as on alterations in the indication for use of particular analgesics determined in the label or package insert, allowed by regulatory authorities such as the Food and Drug Administration (FDA) or European Agency for the Evaluation of Medicinal Products (EMEA).

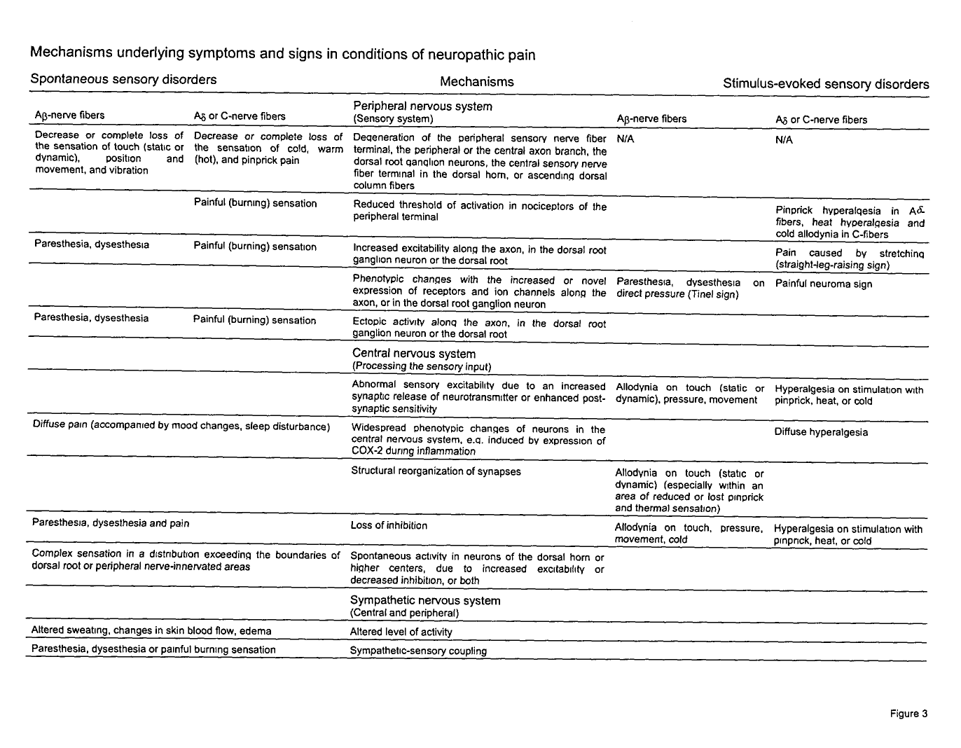

FIG. 3 illustrates specific examples of symptoms (left side) and signs (right side) that underlie different mechanisms of neuropathic pain.

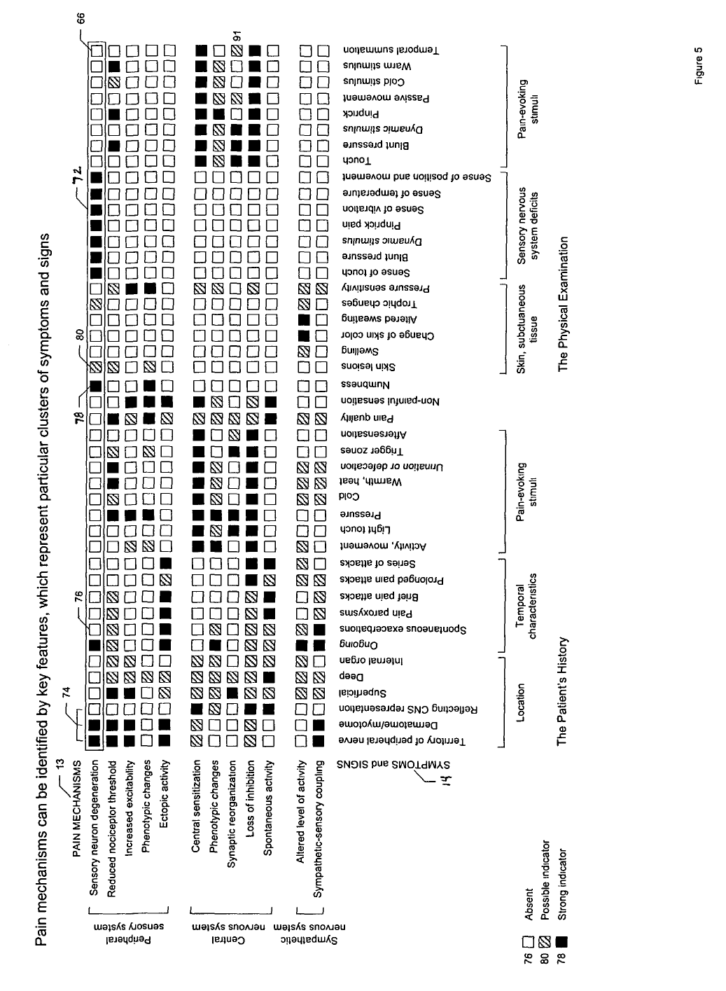

One implementation of mechanism-based pain assessment of pain syndromes is shown schematically in FIGS. 5 and 6. FIG. 6A shows a computer system that can be used to carry out the steps shown in FIG. 6. Initially, a database (70, FIG. 6A) that associates signs and symptoms with pain features is created by investigating the presence and the relative contribution (weighting) of symptoms and signs in a large number of patients with e.g., neuropathic pain. The relationship between single symptoms and signs is examined using statistical methods including correlation analysis, cluster analysis, and analysis of variance. This allows a reduction in the number of symptoms and signs necessary to identify corresponding pain mechanisms. Pain features representing those symptoms and signs, that are attributable to a single mechanism, will be identified by statistical techniques, for example by using an explanatory factor analysis.

As a result, a series of highly specific pain features (66) (each of which is represented by a single row (72) of boxes (74) in FIG. 5) are established, that can be associated with individual pain mechanisms (13) (listed on the left hand side in FIG. 5). Each of these pain features includes a set of single symptoms and signs (14) (each of which is represented by a box (74) in FIG. 5). The symptoms and signs can be grouped in various ways, such as according to the sequence of their investigation in the questionnaire and in the standardized physical examination. In FIG. 5, the absence or presence of a single symptom or sign is depicted graphically as an empty box (76) or a filled box (78), respectively. However, absence or presence may be depicted using any suitable graphical indication such as histograms, lines of different thickness, or different symbols. The relevance of an indicative symptom or sign is given, in this example, by a dashed square (80) for a possible indicator and a black square (78) for a strong indicator (this also can be graphically represented in any of a number of different ways). In the database 70, the relevance can be expressed by numbers according to the assigned rating score.

As discussed above, a patient's pain profile is analyzed to detect the presence of pain features. In a patient whose signs and symptoms are due to the presence of a single pain mechanism, the pain profile is identical to the specific pain feature for that single underlying mechanism. FIG. 5 illustrates pain profiles in hypothetical patients having a single pain feature due to the presence of a single pain mechanism. However, most pain patients will have multiple pain mechanisms operating, and in consequence their pain profile will be a complex composed of multiple pain features whose individual elements may overlap. Here, the database (70) can be used as a “key” to decipher the pain profile “code”.

FIG. 6 illustrates the pain profile (64) of a particular patient with postherpetic neuralgia. Step 3 of FIG. 6 illustrates how, by analyzing the pain profile, three pain features (66), represented by the three rows of boxes in Step 3, can be shown to make up this patient's pain profile. These pain features reflect the concurrence of three different pain mechanisms (13) in this patient that together contribute to the overall pain syndrome (62), i.e., deafferentation pain in this example.

In Step 1, pain-related symptoms are documented in the patient's history and objective signs are investigated by a physical examination under the standardized conditions of a mechanism-based assessment of pain. In Step 2, the information obtained on the absence or presence, and the rating scores of symptoms and signs is transferred into a graphical (as shown in FIG. 6), or numerical, display representing the patient's pain profile (64).

In Step 3, the patient's pain profile is analyzed to determine the presence of pain features. Statistical analysis of the individual patient's pain profile can be used to identify single pain features, and in this way allow determination of how many and which mechanisms are responsible for the pain the patient experiences. As outlined above, a database 70 is used to define reference pain features. Matching the individual patient's pain feature with this reference (the reference pain features shown in FIG. 5) produces an estimated factor score. The estimated factor score can be used to assess the individual patient's standing on the corresponding pain feature in the database and, implicitly, on a single underlying pain mechanism. The higher an estimated factor score, the more likely is the presence of the corresponding pain feature and consequently, pain mechanism. The inclusion of a weighting system using categorical, numerical or visual analogue rating scales substantially improves the ability to assess the relative importance or contribution of a single pain mechanism in the patient's pain syndrome and thus, to determine the predominant pain mechanism(s). In FIG. 6, matches are indicated in Step 3 by larger boxes drawn around the small, symptom-indicating boxes in the top row, and corresponding larger boxes drawn around the matching small boxes in the lower rows. A line is drawn from each of the upper large boxes to a corresponding lower large box to indicate a match.

In the example shown in FIG. 6, there is a good match of a pain identified by this patient with the pain feature of synaptic reorganization (row 91, FIG. 5), which is characterized by evoked pain. Synaptic reorganization within the central nervous system, due to a sprouting of cutaneous A fibers, is characterized by a location of the pain at the surface (indicated by filled-in box 84 in FIG. 6), rather than in deep tissues. Reorganization means that non-nociceptive sensory information from the periphery, e.g., a touch of the skin, is conducted to relay neurons in the central nervous system that under normal conditions receive information only from nociceptive sensory neurons. As a consequence, touch is now erroneously interpreted as pain (indicated by filled-in box 86 in FIG. 6). Since this mechanism involves structures both of the peripheral and the central nervous system, the pain distribution may follow the territory of a peripheral nerve or it may match a distribution reflecting a central nervous system (CNS) pattern. Therefore, these items are not discriminative for this specific mechanism (empty boxes 221 to 223 in FIG. 6). The affected patients will report pain that is evoked by previously non-painful stimuli like light touch or pressure, and they may also describe limited areas of highly increased sensitivity for evoked pain, so-called trigger zones. In correspondence with the descriptions provided in the patient's history, the physician will find that usually non-painful stimuli like touch, blunt pressure or a (dynamic) stimulus moved over the skin cause pain (indicated by filled-in boxes 86, 88 and 90, respectively).

Other signs and symptoms exhibited by this patient, e.g., a decrease in the sense of touch, pinprick, vibration and temperature, correspond with the pattern of sensory deficit that is expected to be seen in the presence of sensory neuron degeneration (85), as revealed by comparison with the corresponding pain feature (66) from the database (i.e., the top row (72) of the iaealized pain profile shown in FIG. 5).

Some of the patient's symptoms, e.g., an ongoing pain (box 93) of an aching quality (box 95), referred to deep tissue (box 97), match with the mechanism of ectopic activity. However, this is not exclusively so, as these symptoms are shared by other mechanisms.

Thus, in this example three mechanisms are present, with sensory neuron degeneration and synaptic reorganization being relatively dominant, and ectopic activity being less relevant. Based on this constellation of pain features, indicating a specific combination of pain mechanisms, the pain syndrome of deafferentation pain is diagnosed in Step 4.

A patient may have more than one pain and this mechanism-based assessment can be performed for each pain to identify if the same or different mechanisms are responsible.

The simple absence or presence of individual signs and symptoms may be insufficient to determine what pain mechanisms are responsible for a patient's pain. For example, two patients may both complain about spontaneous pain and touch-evoked pain, and one of these patients may also complain of activity-evoked pain. Patient A rates his spontaneous pain as high as 6 on the numerical rating scale (NRS) while touch-evoked pain is only slight and reaches not more than NRS 3. Patient A is more affected by activity-evoked pain (NRS up to 5) than touch-evoked pain. Patient B rates his spontaneous pain NRS 4, but suffers from severe touch-evoked pain of NRS up to 10. Activity-evoked pain is absent. In this example, Patient A's pain is due to the presence of phenotypic changes whereas Patient B's predominant pain is due to central sensitization and synaptic reorganization. Thus, to differentiate between pain mechanisms, symptoms and signs have to be weighted to explore the significance of pain mechanisms in individual patients. This example also demonstrates that reaching a conclusion on the presence of a pain feature may require using the complete information about the absence or presence and the rating of all symptoms and signs examined.

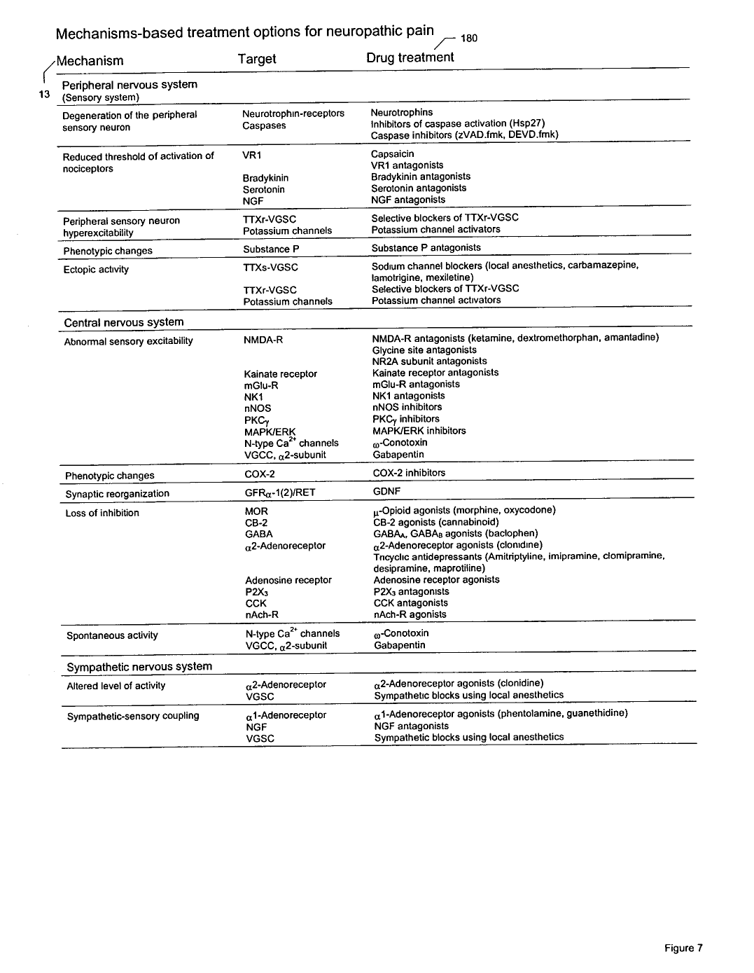

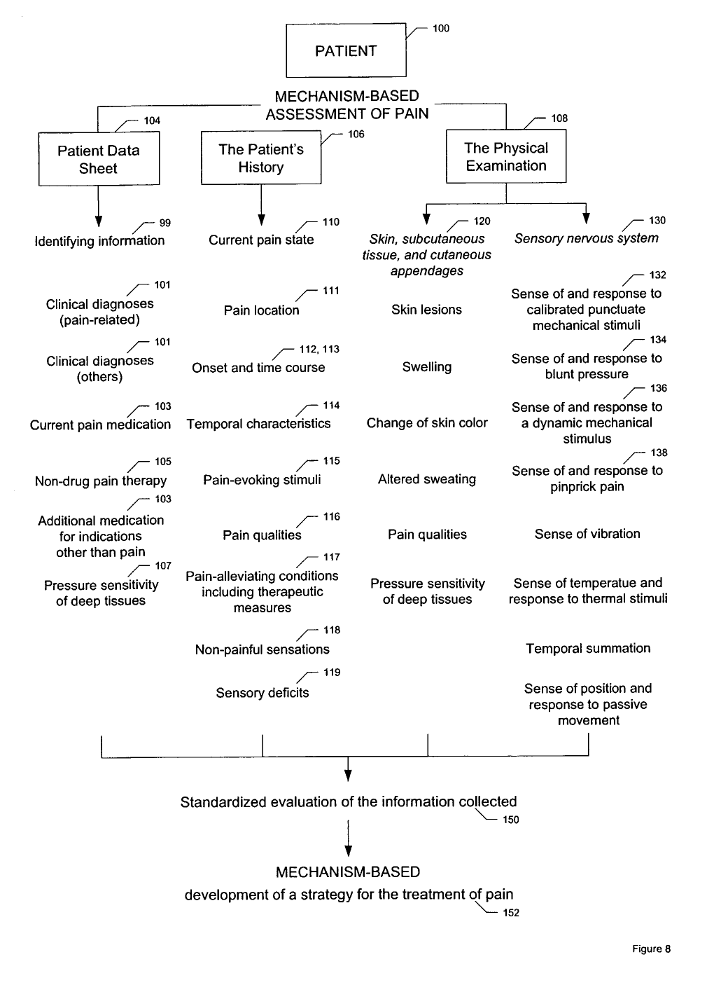

One application of mechanism-based pain assessment is shown schematically in FIGS. 8 and 9. When a patient (100) visits a clinician (102), e.g., a pain specialist, the clinician, or a nurse or assistant (109), fills in a Patient Data Sheet (104), containing, for example, identifying information (99), previous clinical diagnoses (101), medications (103) and therapies (105) previously prescribed, and relevant special investigations (107). Next, the clinician or a nurse or assistant interviews the patient and completes a standardized Patient's History Questionnaire (106) based on the information provided by the patient. The Patient's History Questionnaire, an example of which is shown in FIGS. 11A-11J and discussed below, is designed to include information on the patient's current pain state (110), the location of the pain (111), onset (112), time-course (113), and temporal characteristics (114) of the pain. Other questions relate to how the pain is evoked (115), the quality of the pain (116), factors that tend to alleviate the pain (117), non-painful sensations (118), and sensory deficits (119).

After filling out the Patient's History Questionnaire, the clinician conducts a physical examination (108) of the patient, guided by a standardized Physical Examination Questionnaire, which specifies tests that should be performed. The Physical Examination Questionnaire begins with an assessment of changes in the skin, subcutaneous tissue, and cutaneous appendages (120). However, a main focus of The Physical Examination Questionnaire is the investigation of the nervous system and the sensory system (130) in particular. Guided by the Questionnaire, the clinician thoroughly examines, for example, the patient's responses to specific mechanical stimuli (132) including touch, pressure (134), a brush moved over the surface of the skin to elicit dynamic stimulation of nerve fibers (136), and pinprick (138). The Physical Examination Questionnaire is discussed in more detail below, with reference to FIGS. 12A-12H.

The information obtained by the clinician and the data-taker is stored in a database 71 of patient data and patient profiles (FIG. 6A). The symptoms and signs are used to construct a pain profile for the patient. The pain profile is then analyzed by computer 173 (FIG. 6A) to detect pain features and pain mechanisms, as discussed above. This analysis provides a standardized evaluation of the patient's information (150, FIG. 8). Based on this evaluation, the physician can arrive at (or computer 173 can generate) a mechanism based strategy for treatment of the patient's pain (152, FIG. 8).

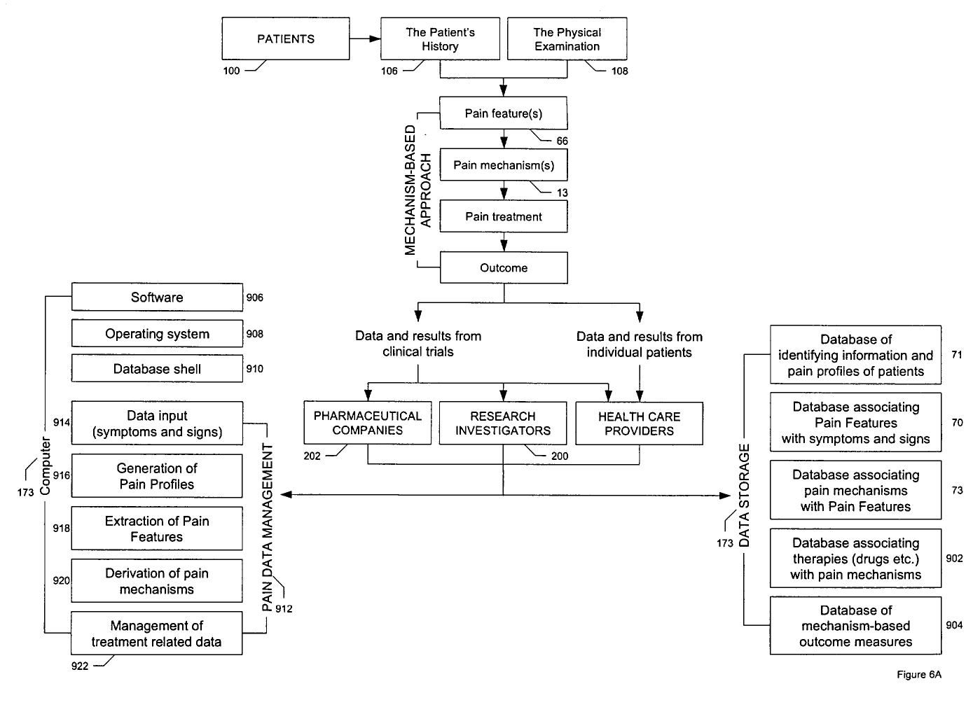

As described above and illustrated in FIGS. 1 and 8, a mechanism-based assessment of pain enables determination of the pain mechanisms present in the patient and use of these mechanisms as targets for the treatment of pain. So far, few treatment options exist that are aimed at the specific mechanisms underlying a patient's pain. Some existing drugs (180) that can be assigned to particular pain mechanisms (13) are listed in FIG. 7. These include e.g., sodium channel blockers like local anesthetics, carbamazepine, and lamotrigine, which reduce ectopic activity. However, knowledge about the effect of drugs on mechanisms involved in the production or maintenance of pain is derived from animal models. Current clinical assessment of pain does not enable identification of pain mechanisms. For example, carbamazepine or lamotrigine are currently prescribed based on a clinical diagnosis, as for trigeminal neuralgia, or empirically to treat brief pain attacks that are similar in terms of duration and intensity to those pain attacks in trigeminal neuralgia. Or they are used as an additive treatment option for neuropathic pain in general, when pain appears resistant to treatment with other drugs. The majority of drugs listed in FIG. 7 are compounds that have not been used in the treatment of patients but represent substances that have been effective in animal models of pain, like inhibitors of MAPK/ERK in conditions associated with an increased excitability of sensory neurons.

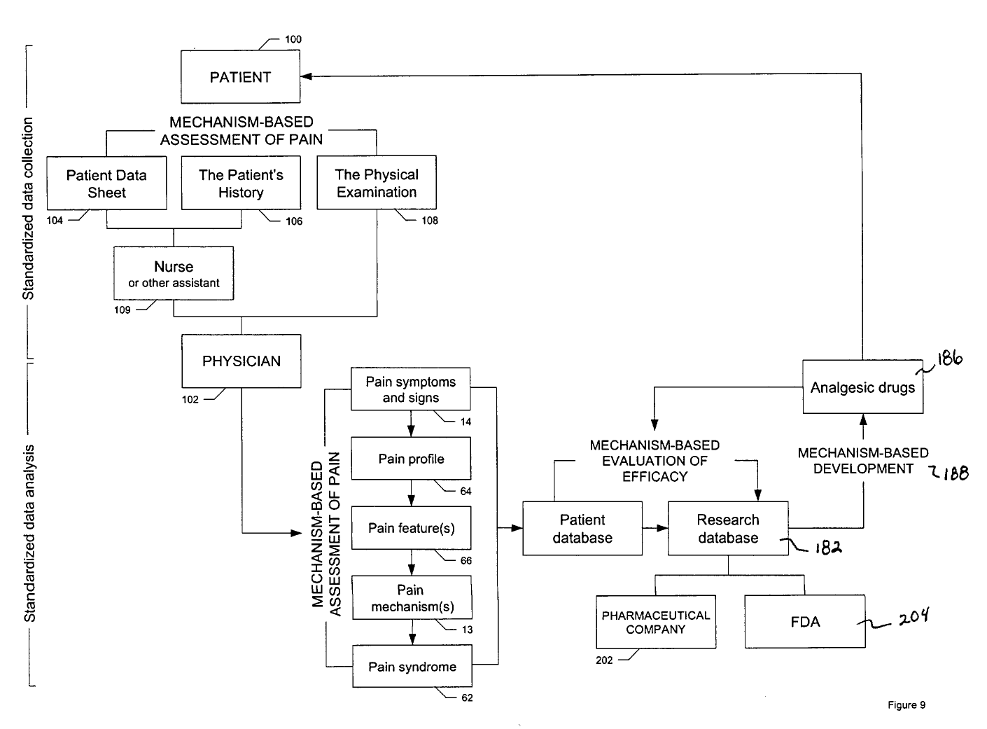

A mechanism-based assessment of pain will improve the transfer of knowledge about the efficacy of compounds derived from animal models into clinical application as it provides a method to design drug trials based on a specific pain mechanism (FIG. 10). Patients may be enrolled into trials only if they have a particular pain feature that is mediated by a particular target for a drug's action. A mechanism-based assessment of pain is required, therefore, to evaluate the specific efficacy of drugs on individual pain mechanisms in a clinical trial (FIG. 10). This is required in order to investigate and compare the effect of both existing analgesic drugs and new analgesics in FIG. 7. If the patient returns for further treatment and follow up care, the Patient's History and Physical Examination Questionnaires can again be used, the updated information stored in the database 70 of patient data and patient profiles and used to compare changes in pain features in the updated pain profile. The evolution or change in the patient's pain profile can be used to monitor changes in the mechanisms responsible for the pain and to determine the susceptibility of the mechanisms to particular treatment strategies. The information obtained during follow up visits can be used to evaluate whether the treatment is in fact reducing the pain, and thus whether the pain medication or dosage should be changed. Because the information is in a standardized format, initial and follow up data can be readily compared. Moreover, data from one patient can be compared to data from other patients, e.g., patients receiving similar pain medication.

Referring to FIGS. 6A and 9-10, with suitable precautions taken to protect patient confidentiality and after informed consent has been obtained from the patients involved, patient data stored in the patient database 180 (FIG. 9), or the derived pain profiles or pain features stored in research database 182 (FIG. 9), may be shared with investigators 200 performing pain-related studies, e.g., studies evaluating the efficacy of pain medications (186), and/or with pharmaceutical companies 202 specializing in the development (188) of pain medications. This shared data may lead to a beneficial exchange of information between the investigators and/or companies and the clinician, with possible benefit to the patient, for example new medications or treatment protocols.

Information obtained from the clinician (and other clinicians) using the Patient's History Questionnaire and the standardized Physical Examination to construct pain profiles and identify pain features, can also be used by pharmaceutical companies in designing new clinical trials performed on participants to evaluate the efficacy of new pain medications. The results of such trials can be stored in research database 182, and forwarded to the FDA (204) in the hope of obtaining approval for the new medications.

The development of new drugs by pharmaceutical companies (202) requires the establishment of biological hypotheses about the mechanisms involved in the pathophysiology of pain (224), e.g. the induction of COX-2 in the central nervous system in conditions of inflammatory pain. This hypothesis is tested in animal models (225), e.g. by administration of COX-2 inhibitors to the central nervous system. A mechanism-based assessment of pain will not only facilitate formulating biological hypotheses, it will also substantially improve the comparability of results obtained in animal studies with findings in drug trials aimed at proofing the concept in patients (phase 2 trials) (226). Information on pain mechanisms, transferred to a research database, can be used by the FDA to classify painful clinical conditions according to their underlying mechanisms (228), and to define new outcome measures (229). Standardized outcome measures represent an important component of guidelines (230) established by the FDA for the development of drugs. These guidelines support the design of large-scale drug trials, that are performed to assess the efficacy and the safety of drugs in diverse clinical conditions (phase 3) (227).

Investigators may use the Patient History and Physical Examination Questionnaires in their own studies and clinical trials. The data obtained from the study may be reported to the FDA, e.g., if the research is being conducted to obtain FDA approval, and/or may be reported in a journal article.

Investigators and pharmaceutical companies may also use information obtained from clinicians who use the Questionnaires to develop other, similar questionnaires and evaluation methods containing different sets of pain features or pain profiles suited to their needs.

FIGS. 11A-11J show one example of a Patient's History Questionnaire that may be used to elicit, it from the patient the information on pain-related symptoms discussed above. Many other types of questionnaires can be developed to elicit patient information.

In the Patient's History Questionnaire (FIGS. 11A-11J), an introductory question (Section Hi., FIG. 11B) explores the patient's current pain state. Subsequently, the Questionnaire asks for the pain location (Section H2, FIG. 11B), which the patient is asked to indicate on an anatomical drawing (FIG. 11C). Many patients describe the presence of more than one pain type. Consequently, the Questionnaire asks the patient to assign the different pain characteristics to a predominant “Pain No. 1” or a less disturbing or distressing “Pain No. 2.” The two types of pain must be distinct in terms of the type of pain sensation, their location or frequency, or their temporal characteristics. If applicable, patients may describe more than two types of pain on separate sheets.

The following section of the Patient's History Questionnaire (Section H3, FIGS. 11B and 11D) investigate the onset and time-course of the pain syndrome, e.g., when the pain started, what seemed to cause it, and whether the pain has changed over time.

In Section H4 (FIGS. 11D-11E), the Questionnaire explores in detail the temporal characteristics of the pain. Questions include, e.g., whether the pain is always present, whether it ever suddenly, spontaneously worsens, how often the pain or an increase in pain is experienced, how long pain attacks last, and whether the patient experiences a series of pain attacks.

In the next section, Section H5 (FIGS. 11E-11G), the Questionnaire asks the patient questions concerning evoked pain, i.e., what events cause the pain. For example, the Questionnaire tries to lead the patient to distinguish between pain evoked by mechanical stimuli, e.g., pressure from touch or clothing, or thermal stimuli, e.g., cold air or a warm bath. The patient is asked to rate the intensity of each specific symptom in order to learn about the respective significance of the symptoms.

In the version of the Questionnaire shown in FIGS. 11A-11, two different rating systems are used to rate the intensity of pain, i.e., categorical (“none, mild, moderate, or severe”) and numerical scales (“rate the intensity on a numerical scale from 0 to 10”). Categorical and numerical scales are currently widely used for global pain ratings. To compare their usefulness in the evaluation of specific pain-related symptoms, both types of scales are included in this version of the Questionnaire. Either or both scales may be used, depending upon which is best accepted by both patients and investigators in a given setting.

In contrast to conventional assessment tools, the Patient History Questionnaire only briefly asks for descriptions of the pain quality (Section H6, FIGS. 11G).

Next, the Questionnaire investigates what drugs, non-drug treatments, and other factors tend to relieve the patient's pain, and what drugs and non-drug treatments have been ineffective (Section H7, FIGS. 11G-11H.) Finally, the Questionnaire investigates sensory disturbances other than pain, e.g., itching, tingling, or paresthesiae, and the presence of numbness (Sections H8 and H9, FIGS. 11H-11I). In Section H9, the patient is also asked to mark any areas of numbness in the anatomical drawing on the following page (FIG. 11J).

Care has been taken to standardize the Patient's History interview. Each item of the Patient's History Questionnaire provides a pre-selected set of answering options. If a patient considers the options inappropriate, the patient is given the opportunity to formulate alternative answers by using her or his own words. Similarly, the Physical Examination Questionnaire (FIGS. 12A-12H) includes a set of standardized questions with pre-selected answering options. Again, the Physical Examination Questionnaire shown in FIGS. 12A-12H is but one example of the many types of questionnaires that can be used to provide a standardized examination. The Physical Examination Questionnaire provides a mechanism-oriented examination that is suitable to be performed both by pain specialists, e.g., clinical pain specialists and investigators in drug trials, and by physicians who do not specialize in pain therapy. To facilitate use by general physicians, the operational instructions for each item of the examination have been kept as simple as possible without losing information.

As discussed above, the Physical Examination Questionnaire begins with an assessment of changes in the skin, subcutaneous tissue, and cutaneous appendages (Section S, FIGS. 12B-12C). Skin changes, such as wounds or scars, may be relevant as they can give a hint to the etiology of a given pain condition. Furthermore, diseases like the complex regional pain syndrome (CRPS) are characterized by swelling, enhanced or reduced sweating, altered skin color and sometimes trophic changes of hairs and nails. Section S also investigates whether the patient is experiencing pressure sensitivity of deep tissues (FIG. 12C). The questions in Section S are formulated as “yes/no” questions, with pre-selected choices of symptoms if “yes” is indicated.

The Physical Examination Questionnaire then examines the nervous system and the sensory system in particular (Section N, FIGS. 12D-12G). Guided by the Questionnaire, the clinician thoroughly examines the patient's responses to specific mechanical stimuli including touch, pressure, a brush moved over the surface of the skin to elicit dynamic stimulation of nerve fibers, and pinprick. Each stimulus is defined in detail to make sure that it will be applied in a standardized fashion. For example, sense of touch is measured by applying two von Freyfilaments, #11 (strength 2.75 g) and #15 (20.9 g), four times to the patient's skin and observing the response. Responses are rated using categorical scales (e.g., “normal”, “decreased sensation”, “painful sensation”). If pain is evoked by a given stimulus, the pain is evaluated in the same manner as in The Patient's History Questionnaire, i.e., by applying both a 4-point categorical scale and a numerical scale ranging from 0 (no pain) to 10 (maximum possible pain).

During the nervous/sensory system evaluation, the clinician also examines the patient's sense of vibration (FIG. 12E), sense of temperature (FIGS. 12E-12F) and sense of position and passive movement (FIGS. 12F-12G). The clinician tests whether the patient's response to repeated administration of calibrated mechanical stimuli changes over time due to temporal summation (FIG. 12F). Optionally, the area affected by relevant findings in any of the items of the Physical Examination Questionnaire may be indicated on an anatomical drawing (FIG. 12H).

Referring again to FIG. 6, in some implementations a data storage 900 holds databases that are created and used by applications running on a computer 173. The databases include a database 71 that contains identifying information and pain profiles of individual patients, a database 70 that associates symptoms and signs and clusters of them with pain features, a database 73 that associates pain mechanisms with pain features and groups of pain features, a database 902 that associates therapies with pain mechanisms, and a database 904 that stores measures of outcome for various pain mechanisms. The software 906 running on computer 173 includes a conventional operating system 908, a database shell 910, and pain data management application modules 912. One module 914 manages the input by a user of symptoms and signs and other information to be stored in the databases or that are otherwise required by the system. A module 916 generates pain profiles from the user data. A module 918 extracts pain features from the pain profiles. A module 920 derives pain mechanisms from the pain features and groups of them. A module 922 provides therapy information based on the derived pain mechanisms.

Although we have described some examples above, other embodiments are also within the scope of the following claims.

What is claimed is:

1. A method comprising
accumulating information from a patient regarding a pain or other sensation experienced by the patient, the information being selected to be indicative of at least one mechanism associated with the pain or sensation, and
analyzing the information to determine one or more pain mechanisms indicated by the information.

2. The method of claim 1 further comprising deriving a pain profile based on the information.

3. The method of claim 2 further comprising analyzing the pain profile to determine the presence of at least one pain feature.

4. The method of claim 3 comprising determining a pain mechanism based on the state of a pain feature.

5. The method of claim 1 wherein the information includes the location of the pain or sensation as indicated on an anatomical diagram.

6. The method of claim 1 in which the information includes the depth of the pain relative to the skin surface of the patient.

7. The method of claim 1 wherein the information pertains to two separate pains and/or sensations the information being selected to be indicative of one or more than one mechanism associated with the pains and/or sensations.

8. The method of claim 1 in which the information includes how long ago the pain or sensation started.

9. The method of claim 1 wherein the information includes the time course of the pain or sensation.

10. The method of claim 9 in which the time course includes changes of the pain or sensation over time.

11. The method of claim 1 where the information includes the temporal characteristics of the pain or sensation.

12. The method of claim 11 in which the temporal characteristics include the time profile of instances of the pain or sensation.

13. The method of claim 1 wherein the information pertains to a sensation other than pain.

14. The method of claim 1 wherein the information pertains to events that evoke a pain or other sensation.

15. The method of claim 14 wherein the events include mechanical stimuli.

16. The method of claim 14 wherein the events include thermal stimuli.

17. The method of claim 1 wherein the information pertains to factors that alleviate a pain or other sensation experienced by the patient.

18. The method of claim 1 in which the information is accumulated using a questionnaire.

19. The method of claim 1 in which the information is accumulated by eliciting the patient's history using standardized questions.

20. The method of claim 1 in which the information is accumulated by performing a physical examination according to a standardized protocol.

21. The method of claim 20 wherein the physical examination includes an assessment of changes in the skin, subcutaneous tissue, and cutaneous appendages.

22. The method of claim 20 wherein the physical examination includes an investigation of the patient's sensory nervous system.

23. The method of claim 22 wherein the investigation includes examining the patient's responses to mechanical stimuli.

24. The method of claim 23 wherein the mechanical stimuli include one or more of the following: touch, pressure, a brush moved over the surface of the skin to elicit dynamic stimulation of nerve fibers, and pinprick.

25. The method of claim 22 wherein the investigation includes examining the patient's sense of temperature.

26. The method of claim 22 wherein the investigation includes examining the patient's sense of vibration.

27. The method of claim 1 further comprising diagnosing a pain syndrome based on the mechanisms determined.

28. The method of c!am 1 further comprising treating the patient based on the mechanisms determined.

29. The method of claim 1 further comprising analyzing the information along with information accumulated from other patients.

30. The method of claim 18 in which the questionnaire includes questions having a pre-selected set of possible answers.

31. The method of claim 1 wherein the information pertains to the quality of the pain.

32. The method of claim 1 wherein the information pertains to a sensory deficit.

33. A method comprising
accumulating information from a patient regarding a pain or other sensation experienced by the patient, the information being selected to be indicative of at least one mechanism associated with the pain or sensation, and
graphically or numerically representing a presence, relative amplitude, and absence of individual symptoms and signs to create a pain profile for the patient.

34. The method of claim 32 further comprising analyzing the pain profile to identify composites of symptoms and signs that represent pain features.

35. A method comprising
accumulating information from a patient regarding a pain or other sensation experienced by the patient, the information being selected to be indicative of at least one mechanism associated with the pain or sensation, and
identifying from the information composites of symptoms and signs that represent pain features that are indicative of a pain mechanism.

36. The method of claim 34 further comprising analyzing the pain features to identify pain mechanisms responsible for the pain.

37. The method of claim 35 further comprising diagnosing a pain syndrome based on the pain mechanisms identified.

38. A method comprising
accumulating information from a patient regarding a pain or other sensation experienced by the patient, the information being selected to be indicative of at least one mechanism associated with the pain or sensation, and
performing a diagnostic or therapeutic evaluation of a patient based on the information.

39. The method of claim 37 also including
at a later time, accumulating additional information from a patient regarding a pain or other sensation experienced by the patient, the information being selected to be indicative of at least one mechanism associated with the pain or sensation, and
performing a diagnostic or therapeutic evaluation of the patient based on the additional information.

40. A method comprising
accumulating information from patients regarding pains or other sensations experienced by the patients, the information being selected to be indicative of at least one mechanism associated with the pains or sensations, and
performing an epidemiological study with respect to the mechanisms associated with the pains or sensations based on the accumulated information.

41. A method comprising
accumulating information from patients regarding pains or other sensations experienced by the patients, the information being selected to be indicative of at least one mechanism associated with the pains or sensations,
selecting or modifying a drug under development based on the accumulated information,
testing the drug on the patients,
accumulating additional information from the patients regarding pains or other sensations experienced by the patients, the information being selected to be indicative of at least one mechanism associated with the pains or sensations, and
iterating the testing and accumulating until a marketable drug is reached.
